(19) United States
(12) Patent Application Publication
(10) Pub. No.: US 2022/0093873 A1
(43) Pub. Date: Mar. 24, 2022

(54) ORGANIC LIGHT EMITTING DEVICE

(71) Applicant: LG CHEM, LTD., Seoul (KR)

(72) Inventors: Ji Hye KIM, Daejeon (KR); Sung Kil HONG, Daejeon (KR); Minseung CHUN, Daejeon (KR); Dong Uk HEO, Daejeon (KR); Yeon Hwan KIM, Daejeon (KR); Sang Duk SUH, Daejeon (KR)

(21) Appl. No.: 17/420,190

(22) PCT Filed: Jan. 31, 2020
(86) PCT No.: PCT/KR2020/001459

§ 371 Date: Jul. 1, 2021

(30) Foreign Application Priority Data
Feb. 1, 2019 (KR) 10-2019-0013443

(51) Int. Cl. H01L 51/00

(52) U.S. Cl. CPC H01L 51/0067; H01L 51/5004; H01L 51/0058; H01L 51/0073

(57) ABSTRACT

Provided is an organic light emitting device having an excellent balance between luminous efficiency and lifespan, the organic light emitting device including:
an anode;
a cathode positioned opposite to the anode;
a light emitting layer positioned between the anode and the cathode;
a hole transport layer positioned between the anode and the light emitting layer; and
an electron transport layer positioned between the light emitting layer and the cathode,
wherein the electron transport layer includes a metal complex compound and an electron transport material having a heterogeneous electron transfer rate constant (K) of 1.2 to 1.65, and
the heterogeneous electron transfer rate constant (K) is calculated by the following Mathematical Equation 1:

$$K = \frac{{k}_{d} + {k}_{a}}{2} \qquad 〈 Mathematical Equation 1 〉$$

wherein in Mathematical Equation 1:
k_{d} (donating k) is an electron donating rate constant and kₐ (accepting k) is an electron accepting rate constant.

### CROSS-REFERENCE TO RELATED APPLICATION(S)

This application is a National Stage Application of International Application No. PCT/KR2020/001459 filed on Jan. 31, 2020, which claims priority to and the benefit of Korean Patent Application No. 10-2019-0013443, filed on Feb. 1, 2019, the disclosure of which is hereby incorporated by reference herein in its entirety.

### FIELD OF THE INVENTION

The present invention relates to an organic light emitting device having an excellent balance between luminous efficiency and lifetime.

### BACKGROUND

In general, an organic light emitting phenomenon refers to a phenomenon where electric energy is converted into light energy by using an organic material. An organic light emitting device using the organic light emitting phenomenon has a wide viewing angle, an excellent contrast, and a fast response time, and has excellent luminance, driving voltage, and response speed characteristics, and thus many studies have proceeded.

The organic light emitting device generally has a structure which includes an anode, a cathode, and an organic material layer interposed between the anode and the cathode. The organic material layer frequently have a multilayered structure that includes different materials in order to enhance efficiency and stability of the organic light emitting device, and for example, the organic material layer can be formed of a hole injection layer, a hole transport layer, a light emitting layer, an electron transport layer, an electron injection layer, etc. In the structure of the organic light emitting device, if a voltage is applied between two electrodes, the holes are injected from an anode into the organic material layer and the electrons are injected from the cathode into the organic material layer, and when the injected holes and electrons meet each other, an exciton is formed, and light is emitted when the exciton falls to a ground state again.

There is a continuing demand for developing new materials for the organic material layer used in the organic light emitting device as described above.

### PRIOR ART LITERATURE

### Patent Literature

(Patent Literature 0001) Korean Patent Publication No. 10-2000-0051826

### SUMMARY

### Technical Problem

There is provided an organic light emitting device having an excellent balance between luminous efficiency and lifetime.

### Technical Solution

To achieve the above object, an organic light emitting device according to the present invention includes:

an anode;

a cathode positioned opposite to the anode;

a light emitting layer positioned between the anode and the cathode;

a hole transport layer positioned between the anode and the light emitting layer; and

an electron transport layer positioned between the light emitting layer and the cathode,

wherein the electron transport layer includes a metal complex compound and an electron transport material having a heterogeneous electron transfer rate constant (K) of 1.2 to 1.65, and

the heterogeneous electron transfer rate constant (K) is calculated by the following Mathematical Equation 1:

$$K = \frac{{k}_{d} + {k}_{a}}{2} \qquad 〈 Mathematical Equation 1 〉$$

wherein in Mathematical Equation 1:

k_{d} (donating k) is an electron donating rate constant and kₐ (accepting k) is an electron accepting rate constant.

### Advantageous Effects

The above-described organic light emitting device can include an electron transport layer including an electron transport material having a heterogeneous electron transfer rate constant (K) value in a specific range, thereby exhibiting an excellent balance between luminous efficiency and lifetime.

### BRIEF DESCRIPTION OF THE DRAWINGS

FIG. 1 illustrates an example of an organic light emitting device including a substrate 1, an anode 2, a hole transport layer 3, a light emitting layer 4, an electron transport layer 5, and a cathode 6;

FIG. 2 illustrates an example of an organic light emitting device including a substrate 1, an anode 2, a hole injection layer 7, a hole transport layer 3, an electron blocking layer 8, a light emitting layer 4, a hole blocking layer 9, an electron transport layer 5, an electron injection layer 10, and a cathode 6;

FIG. 3 shows a current-potential (C-V) curve according to cyclic voltammetry of a compound ETM 1;

FIG. 4 shows a plot of an anodic peak potential (Eₚₐ) versus a scan rate (V/s) obtained from an anodic peak of the C-V curve of the compound ETM 1;

FIG. 5 shows a plot of the anodic peak potential (Eₚₐ) versus ln(v) of the compound ETM 1;

FIG. 6 shows a plot of a cathodic peak potential (E_{pc}) versus a scan rate (V/s) obtained from a cathodic peak of the C-V curve of the compound ETM 1; and

FIG. 7 shows a plot of the cathodic peak potential (E_{pc}) versus ln(v) of the compound ETM 1.

### DETAILED DESCRIPTION

Hereinafter, the present invention will be described in more detail for better understanding.

In the present specification,

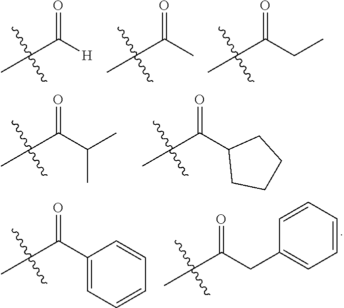

means a bond connected to another substituent.

In the present specification, the term “substituted or unsubstituted” means having no substituents or substituted with one or more substituents selected from the group consisting of deuterium; a halogen group; a cyano group; a nitro group; a hydroxyl group; a carbonyl group; an ester group; an imide group; an amino group; a phosphine oxide group; an alkoxy group; an aryloxy group; an alkylthioxy group; an arylthioxy group; an alkylsulfoxy group; an arylsulfoxy group; a silyl group; a boron group; an alkyl group; a cycloalkyl group; an alkenyl group; an aryl group; an aralkyl group; an aralkenyl group; an alkylaryl group; an alkylamine group; an aralkylamine group; a heteroarylamine group; an arylamine group; an arylphosphine group; or a heteroaryl group containing one or more of N, O, and S atoms, or substituted with a substituent where two or more substituents of the above-exemplified substituents are linked. For example, the “substituent where two or more substituents are linked” can be a biphenyl group. In other words, the biphenyl group can be an aryl group, and can be interpreted as a substituent where two phenyl groups are linked.

In the present specification, the number of carbon atoms in the carbonyl group is not particularly limited, but is preferably 1 to 40 carbon atoms. Specifically, the carbonyl group can be groups having the following structures, but is not limited thereto:

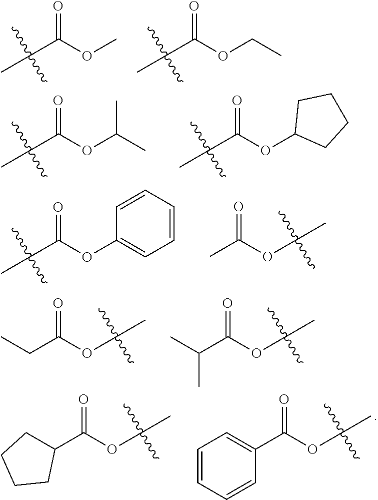

In the present specification, the ester group can have a structure in which oxygen of the ester group can be substituted by a linear, branched, or cyclic alkyl group having 1 to 25 carbon atoms, or an aryl group having 6 to 25 carbon atoms. Specifically, the ester group can be groups having the following structural formulae, but is not limited thereto:

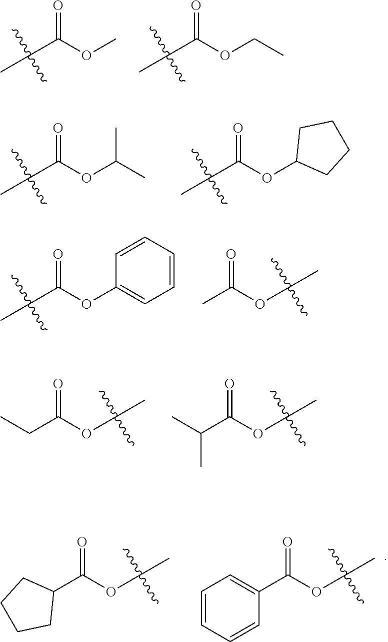

In the present specification, the number of carbon atoms in the imide group is not particularly limited, but is preferably 1 to 25. Specifically, the imide group can be groups having the following structures, but is not limited thereto:

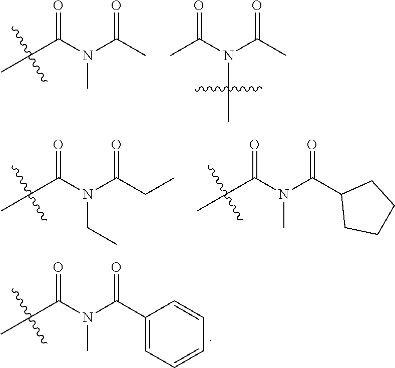

In the present specification, the silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., but is not limited thereto.

In the present specification, the boron group specifically includes a trimethylboron group, a triethylboron group, a t-butyldimethylboron group, a triphenylboron group, a phenylboron group, etc., but is not limited thereto.

In the present specification, examples of the halogen group include fluorine, chlorine, bromine, or iodine.

In the present specification, the alkyl group can be a linear or branched chain, and the number of carbon atoms thereof is not particularly limited, but is preferably 1 to 40. According to one embodiment, the alkyl group has 1 to 20 carbon atoms. According to another embodiment, the alkyl group has 1 to 10 carbon atoms. According to still another embodiment, the alkyl group has 1 to 6 carbon atoms. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, etc., but are not limited thereto.

In the present specification, the alkenyl group can be a linear or branched chain, and the number of carbon atoms thereof is not particularly limited, but is preferably 2 to 40. According to one embodiment, the alkenyl group has 2 to 20 carbon atoms. According to another embodiment, the alkenyl group has 2 to 10 carbon atoms. According to still another embodiment, the alkenyl group has 2 to 6 carbon atoms. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, etc., but are not limited thereto.

In the present specification, the cycloalkyl group is not particularly limited, but the number of carbon atoms thereof is preferably 3 to 60. According to one embodiment, the cycloalkyl group has 3 to 30 carbon atoms. According to another embodiment, the cycloalkyl group has 3 to 20 carbon atoms. According to still another embodiment, the cycloalkyl group has 3 to 6 carbon atoms. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, etc., but are not limited thereto.

In the present specification, the aryl group is not particularly limited, but preferably has 6 to 60 carbon atoms, and can be a monocyclic aryl group or a polycyclic aryl group. According to one embodiment, the aryl group has 6 to 30 carbon atoms. According to one embodiment, the aryl group has 6 to 20 carbon atoms. The aryl group as the monocyclic aryl group can be a phenyl group, a biphenyl group, a terphenyl group, etc., but is not limited thereto. The polycyclic aryl group can include a naphthyl group, an anthracenyl group, a phenanthryl group, a pyrenyl group, a perylenyl group, a chrysenyl group, a fluorenyl group, etc., but is not limited thereto.

In the present specification, the fluorenyl group can be substituted, and two substituent groups can be linked with each other to form a Spiro structure. When the fluorenyl group is substituted,

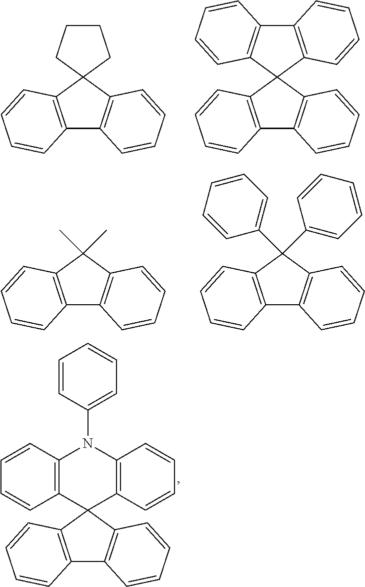

etc. can be formed. However, the structure is not limited thereto.

In the present specification, the heteroaryl group is a heteroaryl group containing one or more of O, N, Si and S as a heteroatom, and the number of carbon atoms thereof is not particularly limited, but is preferably 2 to 60. Examples of the heteroaryl group include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzoimidazole group, a benzothiazole group, a benzocarbazole group, a benzothiophene group, a dibenzothiophene group, a benzofuranyl group, a phenanthroline group, an isoxazolyl group, a thiadiazolyl group, a phenothiazinyl group, a dibenzofuranyl group, etc., but are not limited thereto.

In the present specification, the aryl group in the aralkyl group, the aralkenyl group, the alkylaryl group, and the arylamine group is the same as the aforementioned examples of the aryl group. In the present specification, the alkyl group in the aralkyl group, the alkylaryl group, and the alkylamine group is the same as the aforementioned examples of the alkyl group. In the present specification, the heteroaryl in the heteroarylamines can be applied to the aforementioned description of the heteroaryl group. In the present specification, the alkenyl group in the aralkenyl group is the same as the aforementioned examples of the alkenyl group. In the present specification, the aforementioned description of the aryl group can be applied except that the arylene is a divalent group. In the present specification, the aforementioned description of the heteroaryl group can be applied except that the heteroarylene is a divalent group. In the present specification, the aforementioned description of the aryl group or cycloalkyl group can be applied except that the hydrocarbon ring is not a monovalent group but formed by combining two substituents. In the present specification, the aforementioned description of the heteroaryl group can be applied, except that the heterocycle is not a monovalent group but formed by combining two substituents.

Recently, organic light emitting devices have attracted attention because they have self-light emission and low voltage driving, unlike liquid crystal displays that require a backlight. However, since their efficiency is too low to be applied to a display device requiring light weight and thinness, there has been a demand for a material capable of improving efficiency of organic light emitting devices. However, organic light emitting devices tend to have a reduced lifetime when efficiency increases. Thus, there is a continuous development of materials that increase the efficiency without large lifetime reduction, that is, allow organic light emitting devices to exhibit an excellent balance between efficiency and lifetime.

Accordingly, the present inventors considered that a heterogeneous electron transfer rate constant (K) is suitable as a parameter to understand the electron transport properties of the electron transport material included in the electron transport layer, and they found that an organic light emitting device employing an electron transport material having a heterogeneous electron transfer rate constant (K) value in a specific range exhibits an excellent balance between luminous efficiency and lifetime, thereby completing the present invention.

Furthermore, the electron transport layer includes a metal complex compound together with the electron transport material having a heterogeneous electron transfer rate constant (K) value in a specific range, and the organic light emitting device including such an electron transport layer can have improved luminous efficiency, as compared with an organic light emitting device including an electron transport layer having only the electron transport material, because the metal complex compound induces an increase of dipole moment in the electron transport layer to improve electron injection efficiency from the cathode.

Further, as described later, in order to examine electron mobility of the electron transport material used in the electron transport layer, a heterogeneous electron transfer rate constant value considering both an electron donating rate constant of oxidation reaction and an electron accepting rate constant of reduction reaction is measured, and then only an electron transport material having a heterogeneous electron transfer rate constant value in a specific range is employed, thereby predicting charge transfer between the metal complex compound and the electron transport material in the electron transport layer. Therefore, as compared with an electron transport layer composed of only a single material, bulk electron mobility of the electron transport material and the metal complex compound is maintained at a proper level, thereby contributing to the efficiency increase of organic light emitting device.

Specifically, an organic light emitting device according to one embodiment includes an anode; a cathode positioned opposite to the anode; a light emitting layer positioned between the anode and the cathode; a hole transport layer positioned between the anode and the light emitting layer; and an electron transport layer positioned between the light emitting layer and the cathode, wherein the electron transport layer includes a metal complex compound and an electron transport material having a heterogeneous electron transfer rate constant (K) of 1.2 to 1.65, and the heterogeneous electron transfer rate constant (K) is calculated by the following Mathematical Equation 1:

$$K = \frac{{k}_{d} + {k}_{a}}{2} \qquad 〈 Mathematical Equation 1 〉$$

wherein in Mathematical Equation 1:

k_{d} (donating k) is an electron donating rate constant and kₐ (accepting k) is an electron accepting rate constant,

wherein k_{d} (donating k) of Mathematical Equation 1 is an electron donating rate constant satisfying the following Mathematical Equation 2-1, which is obtained from an anodic peak of a current-potential (C-V) curve according to cyclic voltammetry of the electron transport material:

$$< Mathematical Equation \text{2-1} > \\ {E}_{pa} = {{E}_{a}}^{{0}^{′}} - ( \frac{RT}{α nF} ) ln ( \frac{{RTk}_{d}}{α nF} ) + ( \frac{RT}{α nF} ) ln v$$

wherein in Mathematical Equation 2-1:

Eₚₐ is an anodic peak potential at a maximum current, Eₐ^{0′} is a formal potential at an anodic peak, v is a scan rate, α is an electron transfer coefficient, n is the number of electrons, F is the Faraday constant (96480 C/mol), R is the gas constant (8.314 mol⁻¹K⁻¹), and T is the absolute temperature (298 K);

kₐ (accepting k) is an electron accepting rate constant satisfying the following Mathematical Equation 2-2, which is obtained from a cathodic peak of the C-V curve according to cyclic voltammetry of the electron transport material:

$$< Mathematical Equation \text{2-2} > \\ {E}_{pc} = {{E}_{c}}^{{0}^{′}} + ( \frac{RT}{α nF} ) ln ( \frac{{RTk}_{a}}{α nF} ) - ( \frac{RT}{α nF} ) ln v$$

E_{pc} is a cathodic peak potential at a minimum current, E_{c}^{0′} is a formal potential at a cathodic peak, and v, α, n, F, R, and T are the same as defined in Mathematical Equation 2-1.

Further, the organic light emitting device can further include a hole blocking layer between the light emitting layer and the electron transport layer. When a hole blocking material included in the hole blocking layer exhibits an electron donating rate constant (k_{d}) value in a specific range, as described below, the efficiency of the organic light emitting device can be further improved.

Hereinafter, the present disclosure will be described in detail for each configuration.

### Electron Transport Layer

In general, an electron transport layer in an organic light emitting device refers to a layer that receives electrons from a cathode to transport the electrons to a light emitting layer and blocks movement of holes from an anode to the cathode. Therefore, a material having a lowest unoccupied molecular orbital (LUMO) energy level suitable for easily injecting the injected electrons into the light emitting layer while having a large difference in highest occupied molecular orbital (HOMO) energy level from the light emitting layer such that injection of holes from the light emitting layer to the electron transport layer is prevented is known to be suitable as an electron transport material.

However, HOMO and LUMO energy levels have limitations in determining the electron mobility of the electron transport material, and therefore, even though HOMO and LUMO energy levels of the electron transport material are examined, it is not easy to determine efficiency and/or lifetime tendency of organic light emitting devices therefrom. For this reason, the only method to identify a material capable of increasing the balance between efficiency and lifetime of the organic light emitting device is to examine characteristics of all respective devices by using respective materials as a material for the electron transport layer.

However, in the present disclosure, even though an organic light emitting device employing a specific electron transport material is not manufactured, electron transport properties in the electron transport layer can be determined by examining a heterogeneous electron transfer rate constant (K) value of the electron transport material, and thus the balance between efficiency and lifetime of the device can be easily predicted. Accordingly, by using the determined electron transport material, it is possible to manufacture an organic light emitting device having an excellent balance between efficiency and lifetime.

In this regard, the reason for using the heterogeneous electron transfer rate constant (K) as a parameter to determine the electron transport properties of the electron transport material is that a reaction (reduction reaction) of receiving electrons from the cathode by the electron transport material as an electron acceptor and a reaction (oxidation reaction) of transporting electrons to the light emitting layer by the electron transport material as an electron donor is not a reversible reaction but a quasi-reversible reaction.

Generally, to examine electrochemical behaviors of oxidation and reduction reactions, linear sweep voltammetry (LSV) and cyclic voltammetry (CV) are frequently used. These two methods are common in that a voltage is scanned at a constant rate with respect to a working electrode where the reaction of interest occurs, and the resulting current change is measured. However, of them, cyclic voltammetry (CV) is useful in that whether or not the reaction is reversible can be determined by repeatedly measuring the experiment for each cycle.

In the cyclic voltammetry (CV), in the case of a reversible reaction, oxidation/reduction rates are influenced only by an electron transfer rate, i.e., an electron diffusion rate, and anodic peak potential and cathodic peak potential do not change with the scan rate. Thus, the electron transfer rate in the reversible reaction can be obtained by calculating a diffusion coefficient (D) satisfying the Randles-Sevcik equation of the following Mathematical Equation 3:

$$< Mathematical Equation 3 > \\ {i}_{p} = 0.4463 {nFAC ( \frac{nFvD}{RT} )}^{\frac{1}{2}}$$

wherein in Mathematical Equation 3:

iₚ is a peak current, n is the number of electrons, F is the Faraday constant (96480 C/mol), A is an electrode area, C is a molar concentration, v is a scan rate, R is the gas constant (8.314 mol⁻¹K⁻¹), T is the absolute temperature (298 K), and D is a diffusion coefficient.

In other words, a C-V curve of a subject material is obtained with varying a scan rate, and then the diffusion coefficient (D) can be obtained from the slope of a plot where the x-axis is the square root of the scan rate (v^{1/2}) and the y-axis is the peak current (iₚ).

In contrast, in the case of a quasi-reversible reaction, such as the reaction of the electron transport material in the electron transport layer of the organic light emitting device, the reaction rate is slower than the scan rate, and thus the electron transfer rate may not be obtained from the diffusion rate as in the reversible reaction. Instead, since the anodic and cathodic peaks shift according to the scan rate, the electron transfer rate can be determined by peak shift according to the scan rate. In the present disclosure, the Laviron equation of the following Mathematical Equation 4 was used as an equation for calculating the electron transfer rate constant (k) in the quasi-reversible reaction:

$$< Mathematical Equation 4 > \\ {E}_{p} = {E}^{{0}^{′}} + ( \frac{RT}{α nF} ) ln ( \frac{RTk}{α nF} ) + ( \frac{RT}{α nF} ) ln v$$

wherein in Mathematical Equation 4:

Eₚ is a peak potential, E^{0′} is a formal potential, v is a scan rate, α is an electron transfer coefficient, n is the number of electrons, F is the Faraday constant (96480 C/mol), R is the gas constant (8.314 mol⁻¹K⁻¹), and T is the absolute temperature (298 K).

However, since the electron transfer rate constants, each obtained from anodic peak and cathodic peak of the electron transport material, are different from each other, it is necessary to consider all of them for more accurate examination of the electron transfer rate.

Accordingly, in the present disclosure, the electron transfer rate constant obtained from the anodic peak, i.e., the electron donating rate constant k_{d} (donating k) and the electron transfer rate constant obtained from the cathodic peak, i.e., the electron accepting rate constant kₐ (accepting k) are calculated, respectively, and then a mean value thereof is defined as the heterogeneous electron transfer rate constant (K) of the electron transport material, thereby determining electron transport properties of the electron transport material.

In particular, the properties of electron transfer from the electron transport material to the metal complex compound and the electron transfer properties of the electron transport material transporting electrons from the cathode to the light emitting layer can be identified through the electron transfer rate constant obtained from the anodic peak, i.e., the electron donating rate constant k_{d} (donating k), and the properties of electron transfer from the metal complex compound to the electron transport material can be identified through the electron transfer rate constant obtained from the cathodic peak, i.e., the electron accepting rate constant kₐ (accepting k).

Specifically, the heterogeneous electron transfer rate constant (K) of the electron transport material can be obtained by the following method.

First, the electron transport material is dissolved in dimethylformamide (DMF) at a concentration of 3 mM, and then a current-potential (C-V) curve according to cyclic voltammetry of the electron transport material is obtained with varying the scan rate.

Next, a method of calculating the electron donating rate constant k_{d} (donating k) of the electron transport material is as follows:

a1) From the anodic peak of the C-V curve, a graph where the x-axis is the scan rate (V/s) and the y-axis is the anodic peak potential (Eₚₐ) is plotted. A formal potential (Eₐ^{0′}) value when x is 0 (the scan rate is 0) is obtained therefrom;

b1) Further, from the anodic peak of the C-V curve, a graph where the x-axis is ln(v) and the y-axis is the anodic peak potential (Eₚₐ) is plotted. After fitting the graph to a straight line, the slope and the y-intercept are obtained therefrom;

c1) By using the slope and the y-intercept obtained as above, the electron donating rate constant k_{d} (donating k) satisfying the following Mathematical Equation 2-1 is calculated:

$$< Mathematical Equation \text{2-1} > \\ {E}_{pa} = {{E}_{a}}^{{0}^{′}} - ( \frac{RT}{α nF} ) ln ( \frac{{RTk}_{d}}{α nF} ) + ( \frac{RT}{α nF} ) ln v$$

wherein in Mathematical Equation 2-1:

Eₚₐ is an anodic peak potential at a maximum current, Eₐ^{0′} is a formal potential at an anodic peak, v is a scan rate, α is an electron transfer coefficient, n is the number of electrons, F is the Faraday constant (96480 C/mol), R is the gas constant (8.314 mol⁻¹K⁻¹), and T is the absolute temperature (298 K), and

the y-intercept obtained in the step b1) corresponds to

$${{E}_{a}}^{{0}^{′}} - ( \frac{RT}{α nF} ) ln ( \frac{{RTk}_{d}}{α nF} ) ,$$

and therefore, the formal potential (Eₐ^{0′}) value obtained in the step a1) and the slope

$$( \frac{RT}{α nF} )$$

value obtained in the step b1) can be used to calculate the k_{d} (donating k) value.

Next, a method of calculating the electron accepting rate constant kₐ (accepting k) of the electron transport material is as follows:

a2) From the cathodic peak of the C-V curve, a graph where the x-axis is the scan rate (V/s) and the y-axis is the cathodic peak potential (E_{pc}) is plotted. A formal potential (E_{c}0′) value when x is 0 (the scan rate is 0) is obtained therefrom;

b2) Further, from the cathodic peak of the C-V curve, a graph where the x-axis is ln(v) and the y-axis is the cathodic peak potential (E_{pc}) is plotted. After fitting the graph to a straight line, the slope and the y-intercept are obtained therefrom;

c2) By using the slope and the y-intercept obtained as above, the electron accepting rate constant kₐ (accepting k) satisfying the following Mathematical Equation 2-2 is calculated:

$$< Mathematical Equation \text{2-2} > \\ {E}_{pc} = {{E}_{c}}^{{0}^{′}} + ( \frac{RT}{α nF} ) ln ( \frac{{RTk}_{a}}{α nF} ) - ( \frac{RT}{α nF} ) ln v$$

wherein E_{pc} is a cathodic peak potential at a minimum current, E_{c}^{0′} is a formal potential at a cathodic peak, and v, α, n, F, R, and T are the same as defined in Mathematical Equation 2-1, and

the y-intercept obtained in the step b2) corresponds to

$${{E}_{c}}^{{0}^{′}} + ( \frac{RT}{α nF} ) ln ( \frac{{RTk}_{a}}{α nF} ) ,$$

and therefore, the formal potential (E_{c}^{0′}) value obtained in the step a2) and the slope

$$- ( \frac{RT}{α nF} )$$

value obtained in the step b2) can be used to calculate the kₐ (accepting k) value.

The obtained electron donating rate constant k_{d} (donating k) and electron accepting rate constant kₐ (accepting k) of the electron transport material are averaged as in Mathematical Equation 1 to obtain the heterogeneous electron transfer rate constant (K) of the electron transport material.

Meanwhile, the electron transport layer of the organic light emitting device according to one embodiment includes the metal complex compound and the electron transport material having the heterogeneous electron transfer rate constant (K) of 1.2 to 1.65, as calculated by the above-described method. In this regard, when the heterogeneous electron transfer rate constant (K) of the electron transport material is less than 1.2, charge transfer between the electron transport material and the metal complex compound is not easy, and thus transfer of electrons from the cathode to the light emitting layer is difficult. Accordingly, there is a problem that a driving voltage of the organic light emitting device can increase and the efficiency thereof can decrease. Further, when the heterogeneous electron transfer rate constant (K) of the electron transport material is more than 1.65, excess electrons are transferred to the light emitting layer and thus an electron-hole balance in the light emitting layer can be disturbed. This causes a problem of lifetime reduction of the organic light emitting device.

In contrast, when the organic light emitting device includes the electron transport material having the heterogeneous electron transfer rate constant (K) value in the above-described range, the number of electrons transferred from the cathode to the light emitting layer can be efficiently controlled, and as a result, a balance between luminous efficiency and lifetime can become excellent.

Preferably, the electron transport material having the heterogeneous electron transfer rate constant (K) of 1.2 to 1.65 is a compound of Chemical Formula 1:

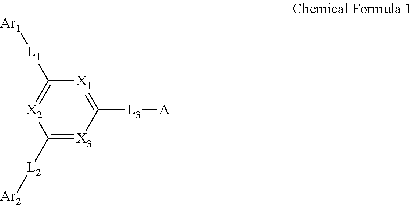

wherein in Chemical Formula 1:

X₁ to X₃ are each independently N or CH, and at least one of X₁ to X₃ is N;

L₁ to L₃ are each independently a single bond or a substituted or unsubstituted C₆₋₆₀ arylene;

Ar₁ and Ar₂ are each independently a substituted or unsubstituted C₆₋₆₀ aryl or a substituted or unsubstituted C₂₋₆₀ heteroaryl containing any one or more heteroatoms selected from the group consisting of N, O, and S;

A is a monovalent substituent derived from a compound of any one of the following Chemical Formulae 2-1 to 2-3:

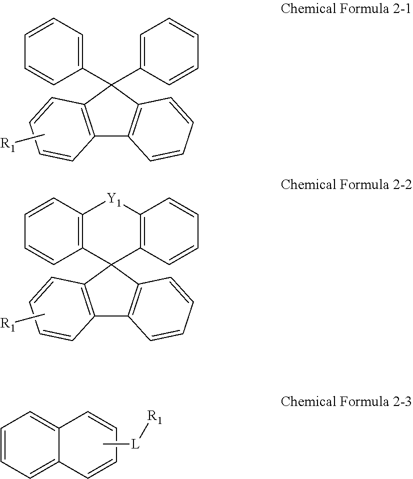

wherein in Chemical Formulae 2-1 to 2-3:

Y₁ is O or S;

L is a C₆₋₆₀ arylene; and

R₁ is hydrogen, deuterium, cyano, a C₆₋₆₀ aryl, or a C₆₋₆₀ aryl substituted with cyano.

Preferably, at least two of X₁ to X₃ are N. More preferably, all of X₁ to X₃ are N; or X₁ and X₂ are N and X₃ is CH.

Preferably, L₁ to L₃ are each independently a single bond or phenylene. Further, L is preferably phenylene, or naphthylene. More preferably, L₁ to L₃ are each independently a single bond, or 1,4-phenylene, L is 1,4-naphthylene.

Preferably, Ar₁ and Ar₂ are each independently phenyl, biphenylyl, terphenylyl, naphthyl, or pyridinyl. More preferably, Ar₁ and Ar₂ are each independently phenyl, biphenylyl, terphenylyl, 2-naphthyl, or pyridin-2-yl.

Preferably, A is any one substituent selected from the group consisting of the following substituents:

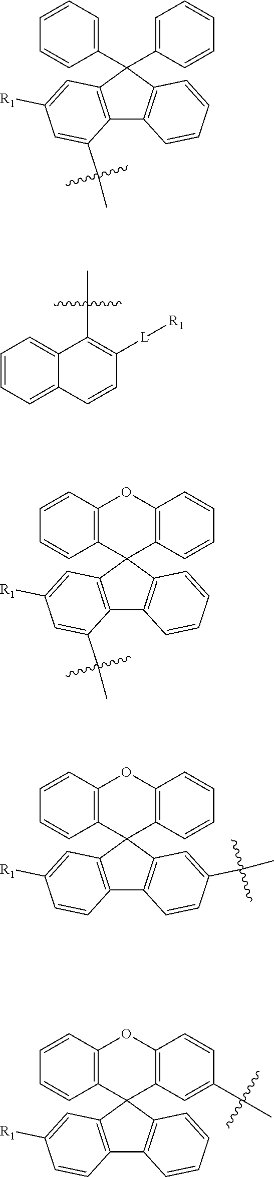

wherein:

L is naphthylene; and

R₁ is hydrogen, cyano, or cyanophenyl.

Representative examples of the electron transport material of Chemical Formula 1 are as follows:

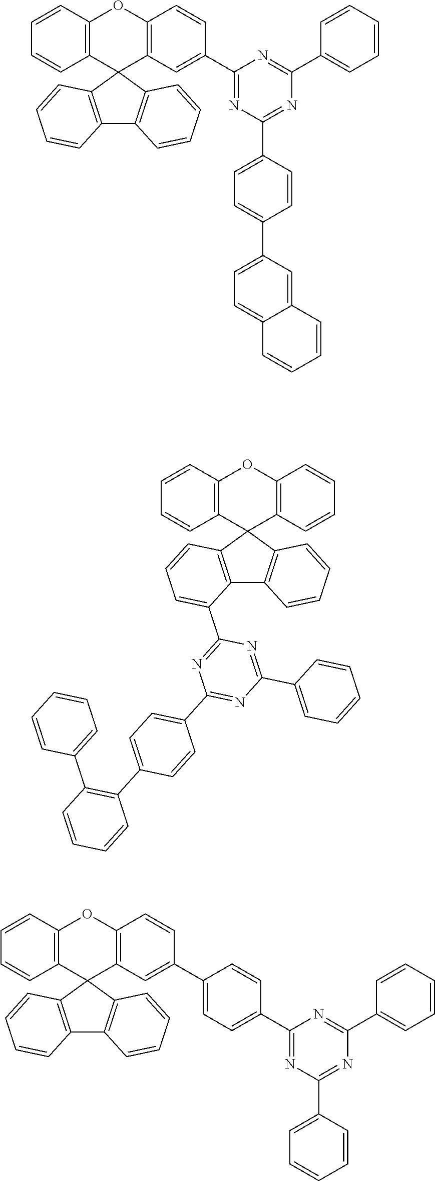 [SMILES: c1ccc(-c2nc(-c3ccc(-c4ccc5ccccc5c4)cc3)nc(-c3ccc4c(c3)C3(c5ccccc5O4)c4ccccc4-c4ccccc43)n2)cc1 ; c1ccc(-c2nc(-c3ccc(-c4ccccc4-c4ccccc4)cc3)nc(-c3cccc4c3-c3ccccc3C43c4ccccc4Oc4ccccc43)n2)cc1 ; c1ccc(-c2nc(-c3ccccc3)nc(-c3ccc(-c4ccc5c(c4)C4(c6ccccc6O5)c5ccccc5-c5ccccc54)cc3)n2)cc1]

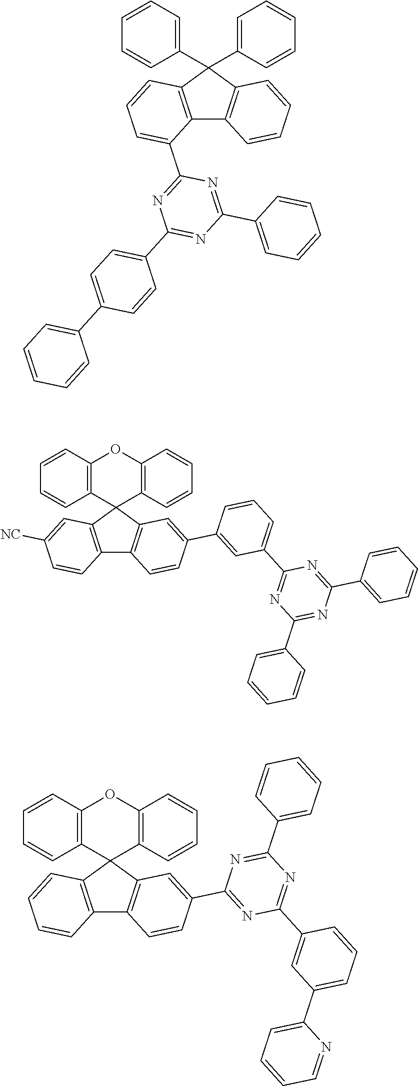 [SMILES: c1ccc(-c2ccc(-c3nc(-c4ccccc4)nc(-c4cccc5c4-c4ccccc4C5(c4ccccc4)c4ccccc4)n3)cc2)cc1 ; N#Cc1ccc2c(c1)C1(c3ccccc3Oc3ccccc31)c1cc(-c3cccc(-c4nc(-c5ccccc5)nc(-c5ccccc5)n4)c3)ccc1-2 ; c1ccc(-c2nc(-c3cccc(-c4ccccn4)c3)nc(-c3ccc4c(c3)C3(c5ccccc5Oc5ccccc53)c3ccccc3-4)n2)cc1]

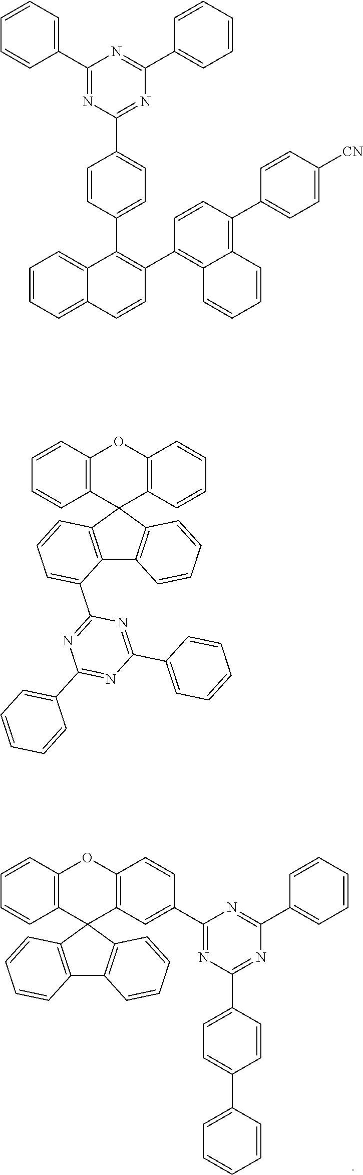 [SMILES: N#Cc1ccc(-c2ccc(-c3ccc4ccccc4c3-c3ccc(-c4nc(-c5ccccc5)nc(-c5ccccc5)n4)cc3)c3ccccc23)cc1 ; c1ccc(-c2nc(-c3ccccc3)nc(-c3cccc4c3-c3ccccc3C43c4ccccc4Oc4ccccc43)n2)cc1 ; c1ccc(-c2ccc(-c3nc(-c4ccccc4)nc(-c4ccc5c(c4)C4(c6ccccc6O5)c5ccccc5-c5ccccc54)n3)cc2)cc1]

Examples of the compound of Chemical Formula 1 can be prepared by a preparation method as in the following Reaction Scheme 1:

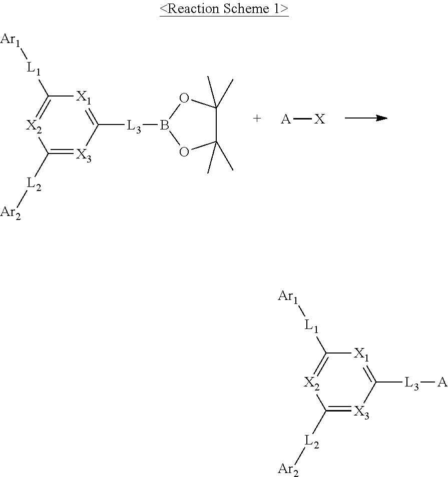

wherein in Reaction Scheme 1, X is halogen, preferably, bromo or chloro, and descriptions of the other substituents are the same as defined in Chemical Formula 1. Specifically, the reaction is a Suzuki coupling reaction, and is preferably performed in the presence of a palladium catalyst. A reactor for the Suzuki coupling reaction can be modified as known in the art. The preparation method can be specified in more detail in Preparation Example below.

Meanwhile, the electron transport layer further includes the metal complex compound, in addition to the electron transport material, wherein the metal complex compound refers to a complex of a metal selected from the group consisting of alkali metals, alkaline earth metals, transition metals, and metals of Group 13 in the periodic table.

Preferably, the metal complex compound can be of the following Chemical Formula 3, wherein M is a central metal, L₁₁ is a main ligand, and L₁₂ is an ancillary ligand:

M(L₁₁)ₙ₁(L₁₂)ₙ₂ <Chemical Formula 3>

wherein in Chemical Formula 3:

M is lithium, beryllium, manganese, copper, zinc, aluminum, or gallium:

L₁₁ is a substituted or unsubstituted 8-hydroxyquinolinato, or a substituted or unsubstituted 10-hydroxybenzo[h]quinolinato;

L₁₂ is halogen, substituted or unsubstituted phenolato, or substituted or unsubstituted naphtholato;

n1 is 1, 2, or 3;

n2 is 0 or 1; and

n1+n2 is 1, 2, or 3,

wherein when n1 is 2 or more, 2 or more of L₁₁ are the same as or different from each other.

More preferably, L₁₁ is halogen or 8-hydroxyquinolinato that is unsubstituted or substituted with C₁₋₄ alkyl; or halogen or 10-hydroxybenzo[h]quinolinato that is unsubstituted or substituted with C₁₋₄ alkyl; and

L₁₂ is halogen. phenolato that is unsubstituted or substituted with C₁₋₄ alkyl, or naphtholato that is unsubstituted or substituted with C₁₋₄ alkyl.

Alternatively, L₁₁ is 8-hydroxyquinolinato, 2-methyl-8-hydroxyquinolinato, or 10-hydroxybenzo[h]quinolinato, and

L₁₂ is chloro, o-cresolato, or 2-naphtholato.

Most preferably, the metal complex compound is any one selected from the group consisting of 8-hydroxyquinolinato lithium (LiQ), bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]-quinolinato)zinc, bis(2-methyl-8-hydroxyquinolinato)chlorogallium, bis(2-methyl-8-hydroxyquinolinato)(o-cresolato)gallium, bis(2-methyl-8-hydroxy-quinolinato)(1-naphtholato)aluminum, and bis(2-methyl-8-hydroxy-quinolinato)(2-naphtholato)gallium.

These metal complex compounds can be prepared by a common method known in the art.

Further, the electron transport layer preferably includes the electron transport material and the metal complex compound at a weight ratio in a range of 70:30 to 30:70. When satisfying the above-described range, charge transfer between the electron transport material and the metal complex compound easily occurs, and at the same time, electron mobility predicted by the heterogeneous electron transfer rate constant of the electron transport material can exhibit high reliability.

### Hole Blocking Layer

The organic light emitting device according to one embodiment can further include a hole blocking layer positioned between the light emitting layer and the electron transport layer. Preferably, the hole blocking layer is positioned in contact with the light emitting layer. The hole blocking layer refers to a layer that confines an exciton formation area to the light emitting layer to increase probability of electron/hole recombination, thereby improving efficiency of the organic light emitting device.

Therefore, a hole blocking material included in the hole blocking layer is required to prevent excess hole transfer and to effectively transfer electrons from the electron transport layer to the light emitting layer. In the present disclosure, to examine hole blocking properties of the hole blocking material, the above-described parameter, electron donating rate constant k_{d} (donating k) is used.

Preferably, the hole blocking layer includes only a hole blocking material having an electron donating rate constant k_{d} (donating k) value in a specific range. In other words, the hole blocking layer is preferably composed of the hole blocking material.

In order to examine the electron transport properties of the electron transport material included in the electron transport layer, charge transfer between the electron transport material and the metal complex compound should be considered to consider the electron donating rate constant k_{d} (donating k) as well as the electron accepting rate constant kₐ (accepting k). However, since the hole blocking material as a single material is included in the hole blocking layer, its electron transport properties can be examined only by considering its role of transporting the electrons from the electron transport layer to the light emitting layer. Therefore, since the electron transfer rate constant k_{d} (donating k) representing electron donating properties of the hole blocking material influences efficiency and lifetime of the organic light emitting device, it is important that the hole blocking material has a specific value of k_{d} (donating k).

Preferably, the hole blocking material has k_{d} (donating k) value of 1.25 to 2.25. Specifically, since the electron transport by the hole blocking material in the hole blocking layer also occurs by a quasi-reversible reaction, the electron transfer rate constant (k) of the electron blocking material can be calculated using the Laviron equation of Mathematical Equation 4, as in the method of calculating k_{d} (donating k) value of the electron transport material.

In other words, k_{d} (donating k) is an electron donating rate constant satisfying Mathematical Equation 2-1, obtained from an anodic peak of a C-V curve according to cyclic voltammetry of the hole blocking material. When k_{d} (donating k) of the hole blocking material is excessively low, electron transport ability thereof is decreased, and thus the number of electrons transported to the light emitting layer is decreased to cause an increase in a driving voltage or a decrease in efficiency. When k_{d} (donating k) of the hole blocking material is excessively high, an electron-hole balance is impaired to cause a problem of lifetime reduction. Accordingly, when a hole blocking material satisfying k_{d} (donating k) value in the above-described range is used, characteristics of an organic light emitting device can be improved.

Preferably, the hole blocking material having k_{d} (donating k) of 1.25 to 2.25 is a compound of Chemical Formula 4:

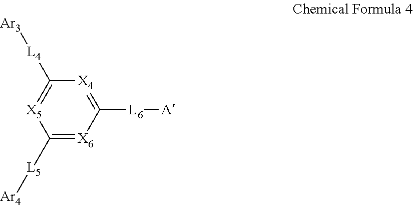

wherein in Chemical Formula 4:

X₄ to X₆ are each independently N or CH, wherein at least one of X₄ to X₆ is N;

L₄ to L₆ are each independently a single bond or a substituted or unsubstituted C₆₋₆₀ arylene;

Ar₃ and Ar₄ are each independently a substituted or unsubstituted C₆₋₆₀ aryl; and

A′ is a monovalent substituent derived from a compound of Chemical Formula 5-1:

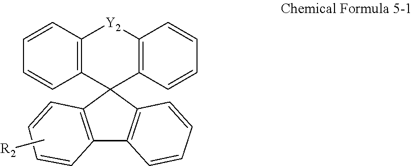

wherein in Chemical Formula 5-1:

Y₂ is O or S; and

R₂ is hydrogen, deuterium, cyano, a C₆₋₆₀ aryl, or a C₆₋₆₀ aryl substituted with cyano.

Preferably, at least two of X₄ to X₆ are N. More preferably, all of X₄ to X₆ are N.

Preferably, L₄ to L₆ are each independently a single bond, phenylene, or biphenyldiyl.

Preferably, Ar₃ and Ar₄ are each independently phenyl, biphenylyl, terphenylyl, or naphthyl.

Preferably, A′ is any one substituent selected from the group consisting of the following substituents:

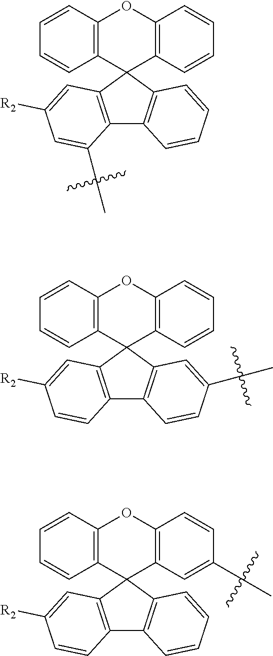

wherein:

R₂ is hydrogen, or cyano.

Representative examples of the hole blocking material of Chemical Formula 4 are as follows:

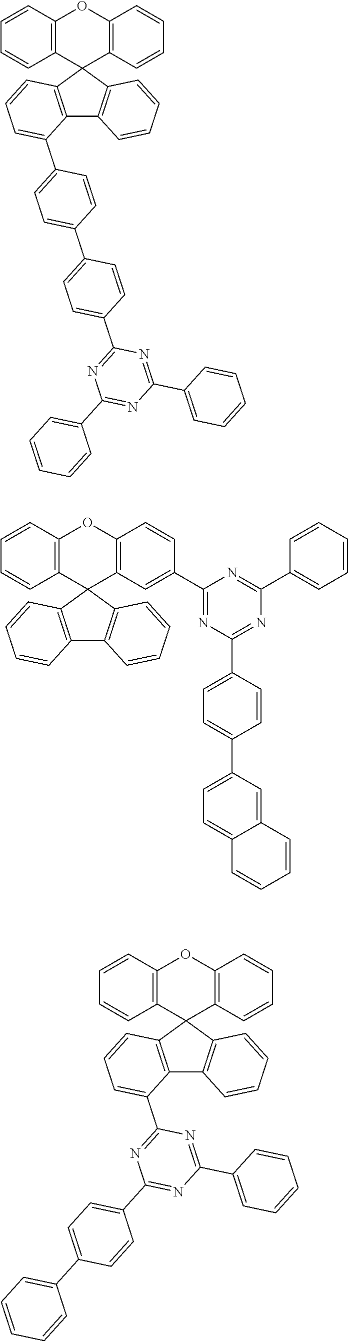 [SMILES: c1ccc(-c2nc(-c3ccccc3)nc(-c3ccc(-c4ccc(-c5cccc6c5-c5ccccc5C65c6ccccc6Oc6ccccc65)cc4)cc3)n2)cc1 ; c1ccc(-c2nc(-c3ccc(-c4ccc5ccccc5c4)cc3)nc(-c3ccc4c(c3)C3(c5ccccc5O4)c4ccccc4-c4ccccc43)n2)cc1 ; c1ccc(-c2ccc(-c3nc(-c4ccccc4)nc(-c4cccc5c4-c4ccccc4C54c5ccccc5Oc5ccccc54)n3)cc2)cc1]

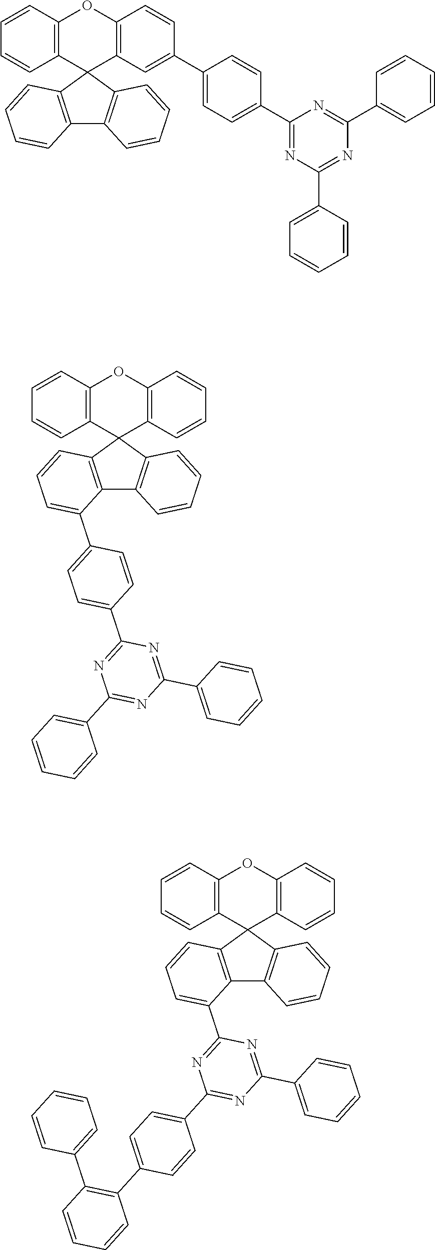 [SMILES: c1ccc(-c2nc(-c3ccccc3)nc(-c3ccc(-c4ccc5c(c4)C4(c6ccccc6O5)c5ccccc5-c5ccccc54)cc3)n2)cc1 ; c1ccc(-c2nc(-c3ccccc3)nc(-c3ccc(-c4cccc5c4-c4ccccc4C54c5ccccc5Oc5ccccc54)cc3)n2)cc1 ; c1ccc(-c2nc(-c3ccc(-c4ccccc4-c4ccccc4)cc3)nc(-c3cccc4c3-c3ccccc3C43c4ccccc4Oc4ccccc43)n2)cc1]

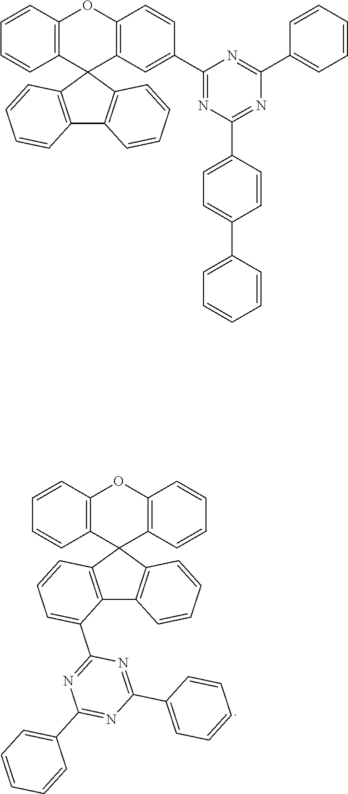 [SMILES: c1ccc(-c2ccc(-c3nc(-c4ccccc4)nc(-c4ccc5c(c4)C4(c6ccccc6O5)c5ccccc5-c5ccccc54)n3)cc2)cc1 ; c1ccc(-c2nc(-c3ccccc3)nc(-c3cccc4c3-c3ccccc3C43c4ccccc4Oc4ccccc43)n2)cc1]

Preferably, the electron transport material and the hole blocking material can be the same as each other.

The compound of Chemical Formula 4 can be prepared by, for example, a preparation method such as the following Reaction Scheme 2:

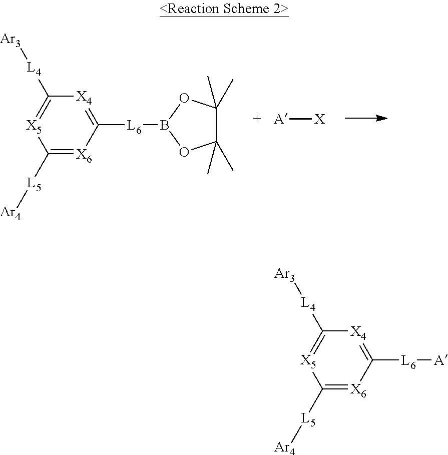

wherein in Reaction Scheme 2, X is halogen, preferably, bromo or chloro, and descriptions of the other substituents are the same as defined in Chemical Formula 4. Specifically, the reaction is a Suzuki coupling reaction, and is preferably performed in the presence of a palladium catalyst. A reactor for the Suzuki coupling reaction can be modified as known in the art. The preparation method can be specified in more detail in Preparation Example below.

Meanwhile, configurations of the organic light emitting device, other than the above-described electron transport layer and hole blocking layer, are not particularly limited, as long as they can be used in organic light emitting devices. Examples thereof can include the following configurations.

### Anode and Cathode

As an anode material, generally, a material having a large work function is preferably used so that holes can be easily injected into the organic material layer. Specific examples of the anode material include metals such as vanadium, chrome, copper, zinc, and gold, or an alloy thereof; metal oxides such as zinc oxide, indium oxide, indium tin oxide (ITO), and indium zinc oxide (IZO); a combination of metals and oxides, such as ZnO:Al or SnO₂:Sb; conductive polymers such as poly(3-methylthiophene), poly[3,4-(ethylene-1,2-dioxy)thiophene](PEDOT), polypyrrole, and polyaniline, and the like, but are not limited thereto.

As a cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

### Hole Injection Layer

The organic light emitting device according to one embodiment can further include, on the anode, a hole injection layer injecting holes from the electrode.

The hole injection layer is composed of a hole injection material, and the hole injection material is preferably a compound which has an ability to transport the holes, a hole injection effect in the anode, and an excellent hole injection effect to the light emitting layer or the light emitting material, prevents movement of an exciton generated in the light emitting layer to the electron injection layer or the electron injection material, and has an excellent thin film forming ability. It is preferable that a highest occupied molecular orbital (HOMO) of the hole injection material is between the work function of the anode material and a HOMO of a peripheral organic material layer.

Specific examples of the hole injection material include metal porphyrine, oligothiophene, an arylamine-based organic material, a hexanitrilehexaazatriphenylene-based organic material, a quinacridone-based organic material, a perylene-based organic material, anthraquinone, polyaniline, and polythiophene-based conductive polymer, etc., but are not limited thereto.

### Hole Transport Layer

The organic light emitting device according to one embodiment can include a hole transport layer that is positioned on the anode or the hole injection layer, and receives holes from the anode or the hole injection layer and transports the holes to the light emitting layer.

The hole transport layer is composed of a hole transport material, and the hole transport material is suitably a material having large mobility to the holes, which can receive holes from the anode or the hole injection layer to transfer the holes to the light emitting layer. Specific examples thereof include an arylamine-based organic material, a conductive polymer, a block copolymer in which a conjugate portion and a non-conjugate portion are present together, etc., but are not limited thereto.

### Electron Blocking Layer

The organic light emitting device according to one embodiment can further include an electron blocking layer between the hole transport layer and the light emitting layer. Preferably, the electron blocking layer is in contact with the light emitting layer, and prevents excess electron transfer to increase probability of hole-electron recombination, thereby improving efficiency of the organic light emitting device. The electron blocking layer includes an electron blocking material. The electron blocking material can include arylamine-based organic materials, but is not limited thereto.

### Light Emitting Layer

The light emitting layer is a layer that emits light in the visible light region by combining holes and electrons, each transported from the hole transport layer and the electron transport layer. The light emitting layer preferably includes a material having high quantum efficiency for fluorescence or phosphorescence. Specifically, the light emitting layer can include a host material and a dopant material.

The host material can include a condensed aromatic ring derivative, a hetero ring-containing compound, etc. Specifically, the condensed aromatic ring derivative includes an anthracene derivative, a pyrene derivative, a naphthalene derivative, a pentacene derivative, a phenanthrene compound, a fluoranthene compound, etc., and the hetero ring-containing compound includes a carbazole derivative, a dibenzofuran derivative, a ladder-type furan compound, a pyrimidine derivative, etc., but are not limited thereto.

The dopant material includes an aromatic amine derivative, a styrylamine compound, a boron complex, a fluoranthene compound, a metal complex, etc. Specifically, the aromatic amine derivative is a condensed aromatic ring derivative having a substituted or unsubstituted arylamino group, and includes a pyrene, an anthracene, a chrysene, a periflanthene, etc., which has an arylamino group, and the styrylamine compound is a compound in which a substituted or unsubstituted arylamine is substituted with at least one arylvinyl group, and one or two or more substituents selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, etc., but are not limited thereto. Further, the metal complex includes an iridium complex, a platinum complex, etc., but is not limited thereto.

### Electron Injection Layer

The organic light emitting device according to the present invention can further include an electron injection layer between the electron transport layer and the cathode. The electron injection layer is a layer that injects electrons from the electrode. A compound which has an ability to transport the electrons, an electron injection effect from the cathode, and an excellent electron injection effect to the light emitting layer or the light emitting material, prevents movement of an exciton generated in the light emitting layer to the hole injection layer, and has an excellent thin film forming ability is preferable.

Specific examples of the materials that can be used for the electron injection layer include LiF, NaCl, CsF, Li₂O, BaO, fluorenone, anthraquinodimethane, diphenoquinone, thiopyran dioxide, oxazole, oxadiazole, triazole, imidazole, perylene tetracarboxylic acid, fluorenylidene methane, anthrone and derivatives thereof, a metal complex compound, a nitrogen-containing 5-membered ring derivative, etc., but are not limited thereto. Here, as the metal complex compound, the above-described metal complex compound which can be used in the electron transport layer can be used. For example, those the same as the metal complex compound used in the electron transport layer can be used, but different metal complex compounds can also be used.

### Organic Light Emitting Device

A structure of the organic light emitting device according to the present disclosure is illustrated in FIG. 1. FIG. 1 illustrates an example of an organic light emitting device including a substrate 1, an anode 2, a hole transport layer 3, a light emitting layer 4, an electron transport layer 5, and a cathode 6. In such a structure, the electron transport material of Chemical Formula 1 and the metal complex compound of Chemical Formula 3 can be included in the electron transport layer 5.

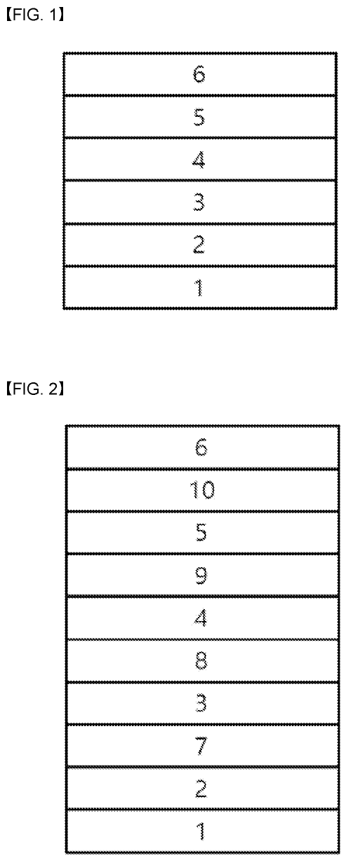

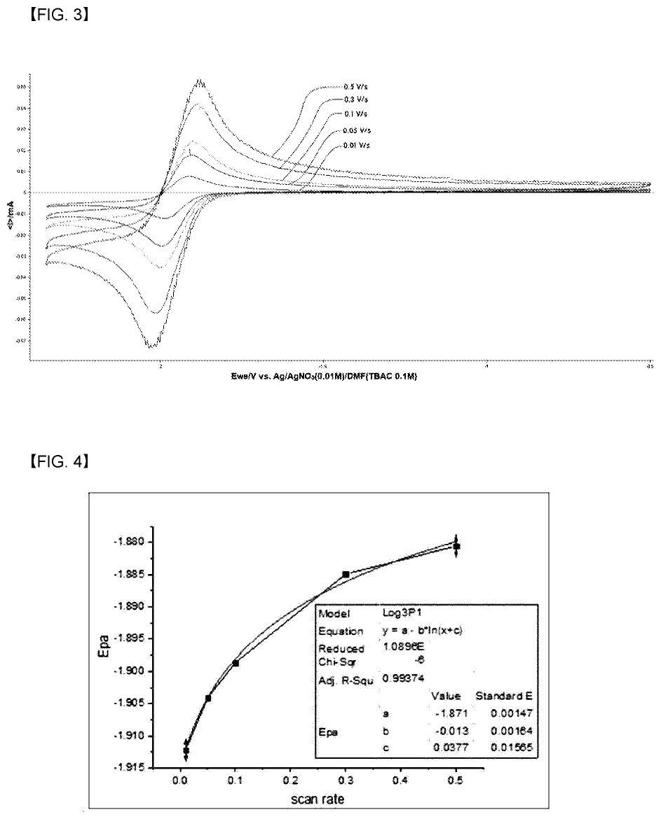

FIG. 2 illustrates an example of an organic light emitting device including a substrate 1, an anode 2, a hole injection layer 7, a hole transport layer 3, an electron blocking layer 8, a light emitting layer 4, a hole blocking layer 9, an electron transport layer 5, an electron injection layer 10, and a cathode 6. In such a structure, the electron transport material of Chemical Formula 1 and the metal complex compound of Chemical Formula 3 can be included in the electron transport layer 5, and the hole blocking material of Chemical Formula 4 can be included in the hole blocking layer 9.

The organic light emitting device according to the present disclosure can be manufactured by sequentially stacking the above-described components. In this case, the organic light emitting device can be manufactured by depositing a metal or a metal oxide having conductivity or an alloy thereof on a substrate to form an anode by using a physical vapor deposition (PVD) method such as sputtering or e-beam evaporation, forming the above-described respective layers thereon, and then depositing thereon a material which can be used as a cathode. In addition to the method described above, an organic light emitting device can be made by sequentially depositing a cathode material, an organic material layer, and an anode material on a substrate. Further, the light emitting layer can be formed by subjecting a host and a dopant to a vacuum deposition method as well as a solution coating method. Here, the solution coating method means spin coating, dip coating, doctor blading, inkjet printing, screen printing, a spray method, roll coating, etc., but is not limited thereto.

In addition to such a method, the organic light emitting device can be manufactured by sequentially depositing a cathode material, an organic material layer, and an anode material on a substrate (WO 2003/012890). However, the manufacturing method is not limited thereto.

Meanwhile, the organic light emitting device according to the present disclosure can be a front side emission type, a back side emission type, or a double side emission type according to the used material.

The manufacturing of the organic light emitting device will be described in detail in the following examples. However, the following examples are presented for illustrative purposes only, and the scope of the present invention is not limited thereto.

### Experimental Example 1: Measurement of Heterogeneous Electron Transfer Rate Constant (K) of Electron Transport Material

A heterogeneous electron transfer rate constant (K) of the following compound ETM 1 was obtained by the following method.

### 1) Plotting of C-V Curve

The compound ETM 1 was dissolved in dimethylformamide (DMF) at a concentration of 3 mM, and then a current-potential (C-V) curve according to cyclic voltammetry was plotted with varying a scan rate (V/s) at 0.01, 0.05, 0.1, 0.3, and 0.5. The curve is shown in FIG. 3. In the C-V curve of FIG. 3, the upward peak is an anodic peak, and the downward peak is a cathodic peak.

### 2) Calculation of Electron Donating Rate Constant k_{d} (Donating k)

a1) From the anodic peak of the C-V curve obtained in step 1), an anodic peak potential (Eₚₐ) value was obtained with respect to each scan rate (V/s) of 0.01, 0.05, 0.1, 0.3, and 0.5, and then shown in the following Table 1. A graph where the x-axis is the scan rate (V/s) and the y-axis is the anodic peak potential (Eₚₐ) was plotted, and shown in FIG. 4. After fitting to the equation of FIG. 4, a, b, and c parameters were obtained. A formal potential (Eₐ^{0′}) value when x is 0 (the scan rate is 0) was obtained therefrom. The obtained formal potential (Eₐ^{0′}) was −1.914 V.

TABLE 1

| Scan rate (V/s) | Eₚₐ |
|---|---|
| 0.01 | −1.91219 |
| 0.05 | −1.90413 |
| 0.1 | −1.89866 |
| 0.3 | −1.88492 |
| 0.5 | −1.88056 |

b1) By using the anodic peak potential (Eₚₐ) value according to each scan rate obtained in the step a1), a graph where the x-axis is ln(v) and the y-axis is the anodic peak potential (Eₚₐ) was plotted, and shown in FIG. 5. After drawing a trend line on the graph of FIG. 5 and fitting to a straight line, the slope and the y-intercept were obtained. The obtained slope and y-intercept were 0.0084 and −1.8764, respectively.

c1) By using the formal potential (Eₐ^{0′})) value obtained in step a1) and the slope and the y-intercept obtained in the step b1), the electron donating rate constant k_{d} (donating k) satisfying Mathematical Equation 2-1 was calculated. Specifically, the y-intercept corresponds to

$${{E}_{a}}^{{0}^{′}} - ( \frac{RT}{α nF} ) ln ( \frac{{RTk}_{d}}{α nF} ) ,$$

and therefore, the formal potential (Eₐ^{0′}) value obtained in the step a1) and the slope

$$( \frac{RT}{α nF} )$$

value obtained in the step b1) were put to calculate the k_{d} (donating k) value. The obtained k_{d} (donating k) was 1.4178.

### 3) Calculation of Electron Accepting Rate Constant kₐ (Accepting k)

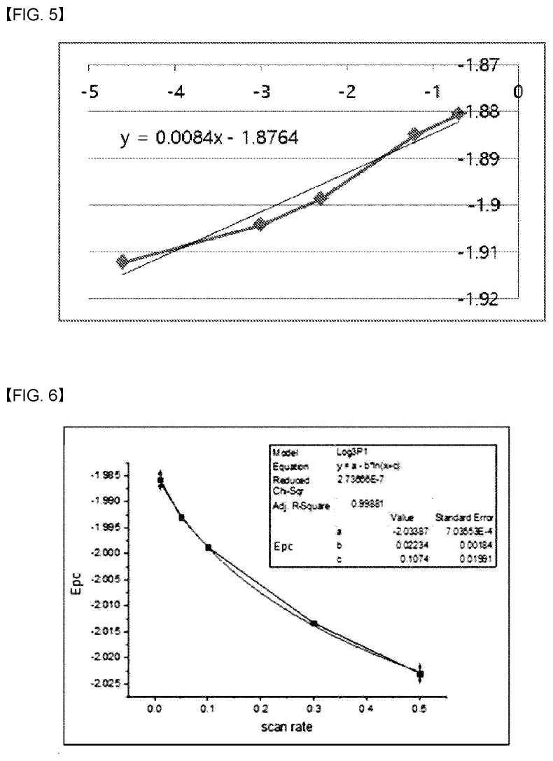

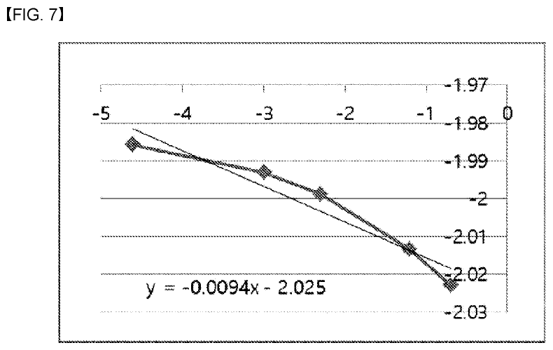

a2) From the cathodic peak of the C-V curve obtained in step 1), a cathodic peak potential (E_{pc}) value was obtained with respect to each scan rate (V/s) of 0.01, 0.05, 0.1, 0.3, and 0.5, and then shown in the following Table 2. A graph where the x-axis is the scan rate (V/s) and the y-axis is the cathodic peak potential (E_{pc}) was plotted, and the graph is shown in FIG. 6. After fitting to the equation of FIG. 6, a, b, and c parameters were obtained. A formal potential (E_{c}^{0′}) value when x is 0 (the scan rate is 0) was obtained. The obtained formal potential (E_{c}^{0′}) was −1.9838 V.

TABLE 2

| Scan rate (V/s) | E_{pc} |
|---|---|
| 0.01 | −1.98577 |
| 0.05 | −1.993 |
| 0.1 | −1.99872 |
| 0.3 | −2.01334 |
| 0.5 | −2.02301 |

b2) By using the cathodic peak potential (E_{pc}) value according to each scan rate obtained in the step a2), a graph where the x-axis is ln(v) and the y-axis is the cathodic peak potential (E_{pc}) was plotted, and the graph is shown in FIG. 7. After drawing a trend line on the graph of FIG. 7 and fitting to a straight line, the slope and the y-intercept were obtained. The obtained slope and y-intercept were −0.0094 and −2.025, respectively.

c2) By using the formal potential (E_{c}^{0′}) value obtained in step a2) and the slope and the y-intercept obtained in the step b2), the electron accepting rate constant kₐ (accepting k) satisfying Mathematical Equation 2-2 was calculated. Specifically, the y-intercept corresponds to

$${{E}_{c}}^{{0}^{′}} + ( \frac{RT}{α nF} ) ln ( \frac{{RTk}_{a}}{α nF} ) ,$$

and therefore, the formal potential (E_{c}^{0′}) value obtained in the step a2) and the slope

$$- ( \frac{RT}{α nF} )$$

value obtained in the step b2) were put to calculate the kₐ (accepting k) value. The obtained kₐ (accepting k) was 1.3330.

### 4) Calculation of Heterogeneous Electron Transfer Rate Constant (K)

The heterogeneous electron transfer rate constant (K) of the compound ETM 1 was calculated using k_{d} (donating k) obtained in the step 2) and kₐ (accepting k) obtained in the step 3) according to Mathematical Equation 1, and as a result, the value was 1.3754.

Heterogeneous electron transfer rate constant (K) was calculated for each of the following ETM 2 to ETM 9 and comparative compounds X1 and X2 in the same manner as the compound ETM 1, and the results are shown in Table 3.

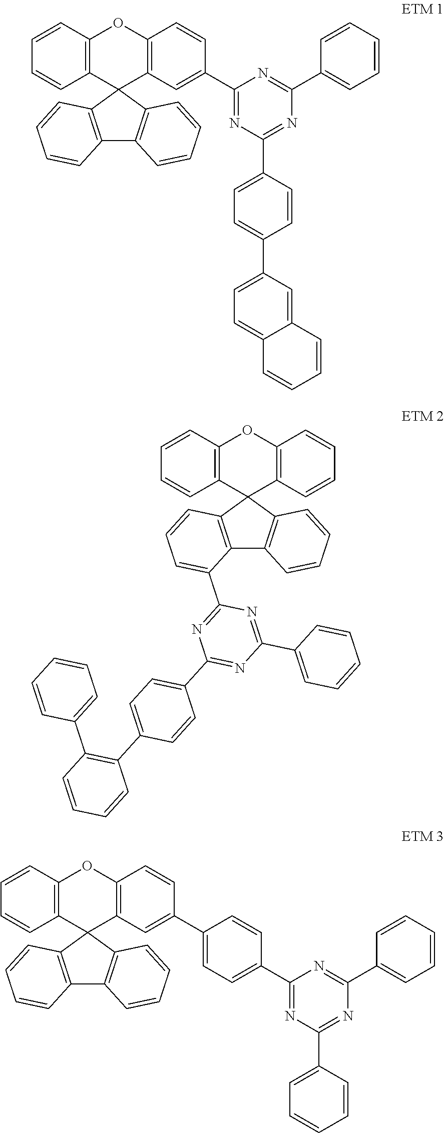

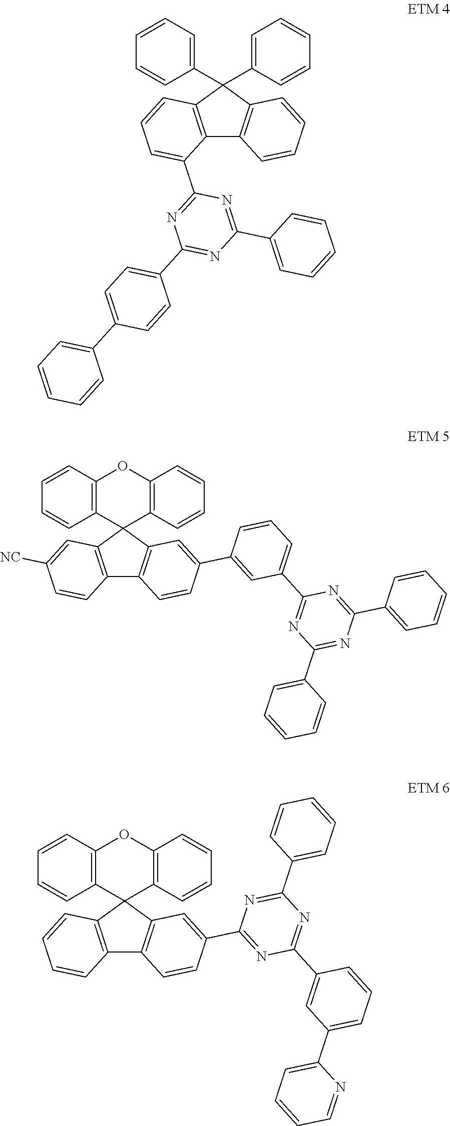 [SMILES: c1ccc(-c2ccc(-c3nc(-c4ccccc4)nc(-c4cccc5c4-c4ccccc4C5(c4ccccc4)c4ccccc4)n3)cc2)cc1 ; N#Cc1ccc2c(c1)C1(c3ccccc3Oc3ccccc31)c1cc(-c3cccc(-c4nc(-c5ccccc5)nc(-c5ccccc5)n4)c3)ccc1-2 ; c1ccc(-c2nc(-c3cccc(-c4ccccn4)c3)nc(-c3ccc4c(c3)C3(c5ccccc5Oc5ccccc53)c3ccccc3-4)n2)cc1]

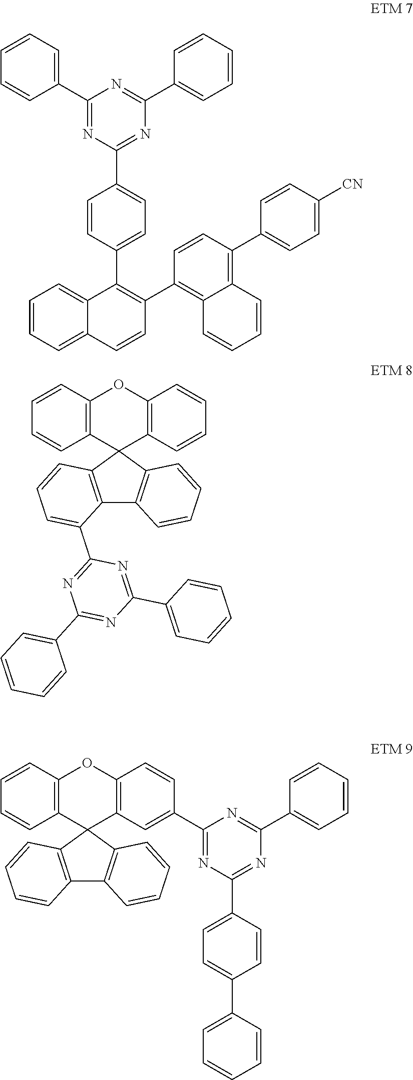

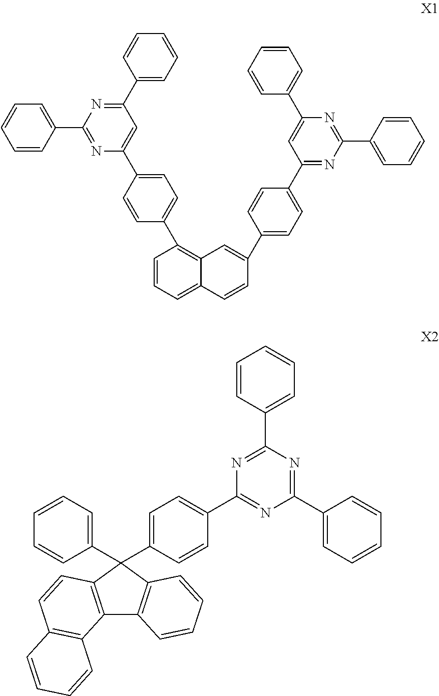 [SMILES: c1ccc(-c2cc(-c3ccc(-c4ccc5cccc(-c6ccc(-c7cc(-c8ccccc8)nc(-c8ccccc8)n7)cc6)c5c4)cc3)nc(-c3ccccc3)n2)cc1 ; c1ccc(-c2nc(-c3ccccc3)nc(-c3ccc(C4(c5ccccc5)c5ccccc5-c5c4ccc4ccccc54)cc3)n2)cc1]

TABLE 3

| | k_{d} | kₐ | |
|---|---|---|---|
| Compound | (donating k) | (accepting k) | K |
| X1 | 1.2220 | 0.6561 | 0.9391 |
| ETM 1 | 1.4178 | 1.333 | 1.3754 |
| ETM 2 | 1.8453 | 0.932 | 1.3887 |
| ETM 3 | 1.7641 | 1.056 | 1.41 |
| ETM 4 | 1.8733 | 0.9686 | 1.421 |
| ETM 5 | 1.5644 | 1.2905 | 1.4275 |
| ETM 6 | 1.226 | 1.6512 | 1.4386 |
| ETM 7 | 1.9396 | 1.1778 | 1.5587 |
| ETM 8 | 2.1158 | 1.0529 | 1.5844 |
| ETM 9 | 1.8464 | 1.3496 | 1.598 |
| X2 | 2.4358 | 0.9480 | 1.6918 |

### Experimental Example 2: Calculation of Electron Donating Rate Constant k_{d} (Donating k) of Hole Blocking Material

Among the compounds HBM 1 to HBM 8 and the comparative compounds Y1 and Y2, the compounds, of which electron donating rate constant k_{d} (donating k) was not obtained in Experimental Example 1, were subjected to calculating electron donating rate constant k_{d} (donating k) in the same manner as the compound ETM 1 of Experimental Example 1, and the results are shown in Table 4.

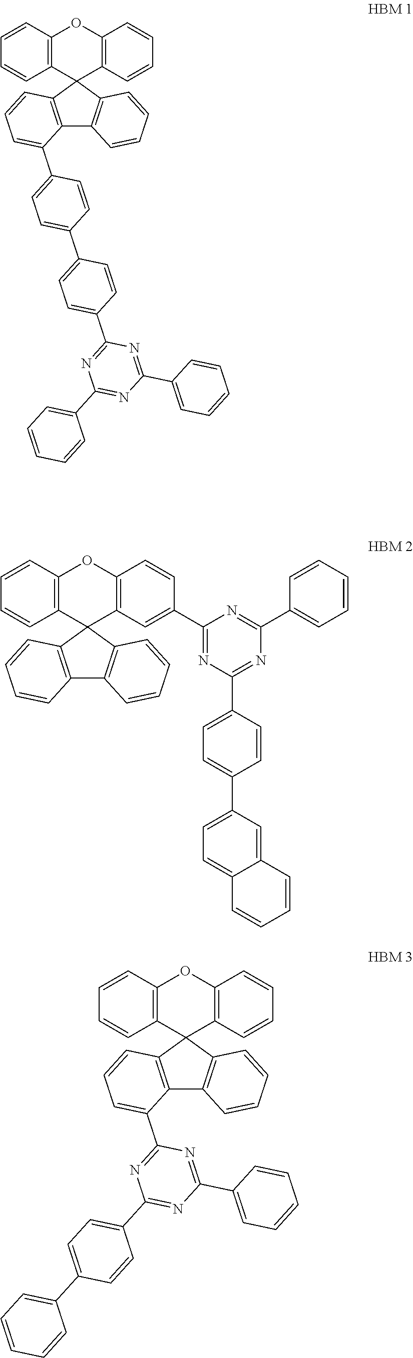 [SMILES: c1ccc(-c2nc(-c3ccccc3)nc(-c3ccc(-c4ccc(-c5cccc6c5-c5ccccc5C65c6ccccc6Oc6ccccc65)cc4)cc3)n2)cc1 ; c1ccc(-c2nc(-c3ccc(-c4ccc5ccccc5c4)cc3)nc(-c3ccc4c(c3)C3(c5ccccc5O4)c4ccccc4-c4ccccc43)n2)cc1 ; c1ccc(-c2ccc(-c3nc(-c4ccccc4)nc(-c4cccc5c4-c4ccccc4C54c5ccccc5Oc5ccccc54)n3)cc2)cc1]

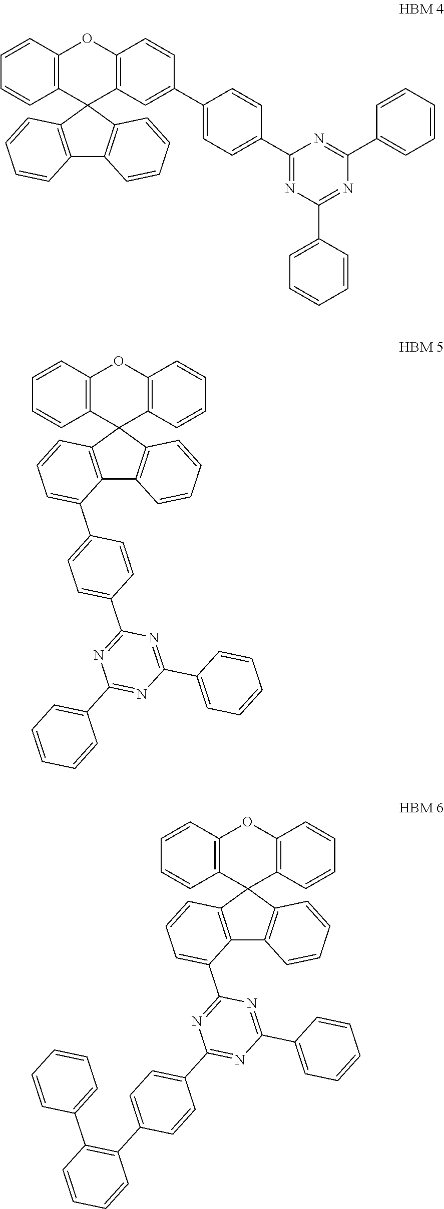 [SMILES: c1ccc(-c2nc(-c3ccccc3)nc(-c3ccc(-c4ccc5c(c4)C4(c6ccccc6O5)c5ccccc5-c5ccccc54)cc3)n2)cc1 ; c1ccc(-c2nc(-c3ccccc3)nc(-c3ccc(-c4cccc5c4-c4ccccc4C54c5ccccc5Oc5ccccc54)cc3)n2)cc1 ; c1ccc(-c2nc(-c3ccc(-c4ccccc4-c4ccccc4)cc3)nc(-c3cccc4c3-c3ccccc3C43c4ccccc4Oc4ccccc43)n2)cc1]

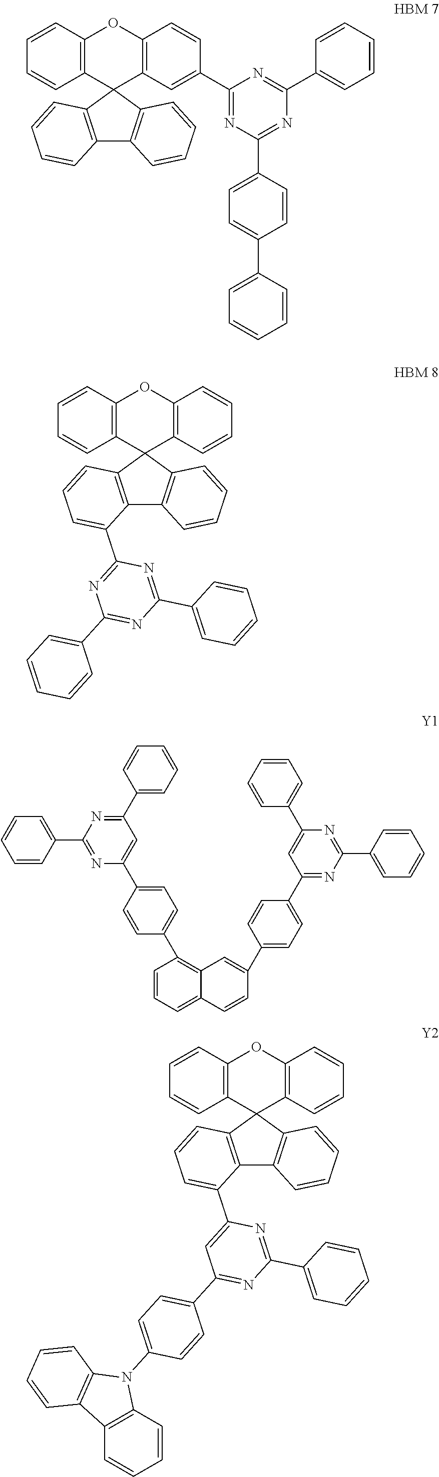

TABLE 4

| | k_{d} |
|---|---|
| Compound | (donating k) |
| Y1 | 1.2220 |
| HBM 1 | 1.3685 |
| HBM 2 | 1.4178 |
| HBM 3 | 1.5644 |
| HBM 4 | 1.5993 |
| HBM 5 | 1.7641 |
| HBM 6 | 1.8571 |
| HBM 7 | 1.8453 |
| HBM 8 | 1.8464 |
| Y2 | 2.7261 |

### <Manufacture of Organic Light Emitting Device>

### Comparative Example 1-1

As an anode, a substrate on which ITO was deposited at 30 Å was cut to a size of 50 mm×50 mm×0.5 mm, and immersed in distilled water in which a detergent was dissolved, and washed by ultrasonic waves. As a detergent, a product available from Fisher Co. was used. As the distilled water, distilled water filtered twice by using a filter available from Millipore Co. was used. The ITO was washed for 30 minutes, and ultrasonic washing was then repeated twice for 10 minutes by using distilled water. After the completion of washing with distilled water, ultrasonic washing was performed using isopropyl alcohol, acetone, and methanol solvent in this order, followed by drying.

A compound HTL1 and P1 were vacuum-deposited at a weight ratio of 97:3 on the prepared anode to form a hole injection layer in a thickness of 106 A. Then, the compound HTL1 was vacuum-deposited on the hole injection layer in a thickness of 1000 Å to form a hole transport layer. Then, a compound HTL2 was vacuum-deposited on the hole transport layer in a thickness of 40 Å to form an electron blocking layer.

Next, a host BH and a dopant BD were vacuum-deposited at a weight ratio of 97:3 on the electron blocking layer to form a light emitting layer in a thickness of 190 Å.

Then, a hole blocking material ETL1 was deposited on the light emitting layer in a thickness of 50 Å to form a hole blocking layer. Then, electron transport material X1 and LiQ were vacuum-deposited at a weight ratio of 50:50 to form an electron transport layer in a thickness of 250 Å. Subsequently, LiQ with a thickness of 7 Å was used to form an electron injection layer, and magnesium and silver (10:1) were used to form a cathode in a thickness of 100 Å. A capping layer (CPL) was deposited in a thickness of 800 Å to complete a device.

In the above process, a deposition rate of the organic material was maintained at 1 Å/sec. At this time, the vacuum deposition of each layer was performed using a cluster-type 1.0E-7 vacuum evaporator (manufactured by Selcos).

### Comparative Example 1-2 and Examples 1-1 to 1-9

Each organic light emitting device was manufactured in the same manner as in Comparative Example 1-1, except that each of materials described in the following Table 5 was used as the electron transport material.

The compounds used in Examples and Comparative Examples are the same as follows:

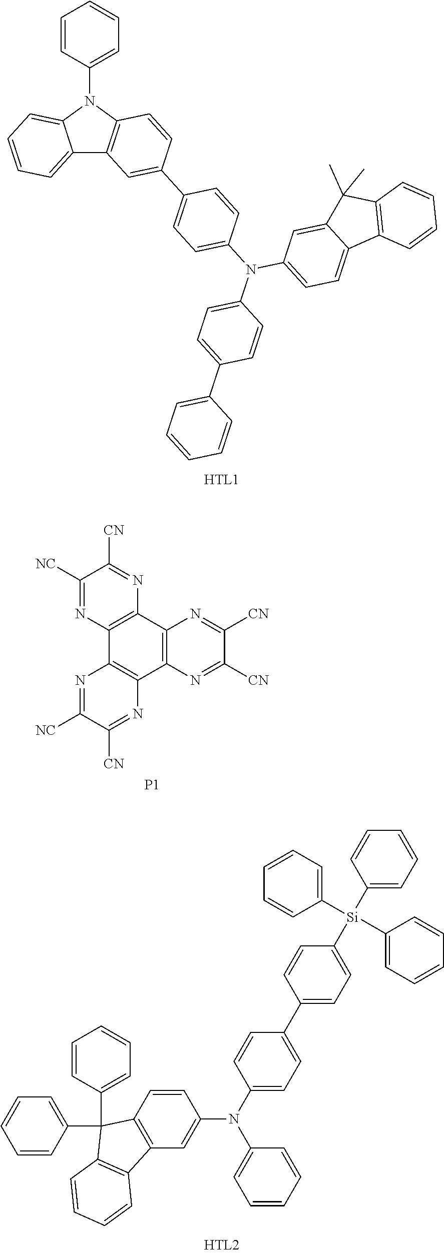

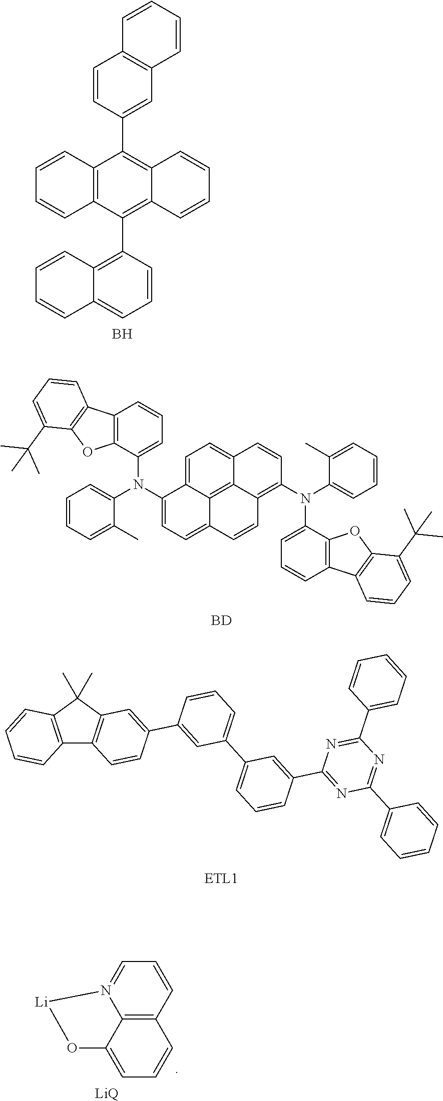

### Experimental Example 3: Characterization of Device According to K Value of Electron Transport Material

Current efficiency and lifetime (T95) were measured using PR-655 IVL available from Photo Research, when current was applied to the organic light emitting devices manufactured in Examples and Comparative Examples, and the results are shown in the following Table 5. At this time, lifetime (T95) means the time to be taken until the luminance reaches 95% of the initial luminance.

Further, to evaluate a balance between current efficiency and lifetime of the organic light emitting devices according to K values of the electron transport materials, evaluation criteria (α) was calculated by considering ranges of the efficiency and lifetime values as in the following Equation 1, and the results are shown in the following Table 5.

Evaluation criteria (α)=current efficiency+(lifetime)/100 <Equation 1>

TABLE 5

| | Kind of | K value of | Current | | Evalu- |
|---|---|---|---|---|---|
| | electron | electron | efficiency | T95 | ation |
| | transport | transport | (cd/A@10 | (hr@950 | criteria |
| | material | material | mA/cm²) | nit) | (α) |
| Comparative | X1 | 0.9391 | 7.42 | 156 | 8.98 |
| Example 1-1 | | | | | |
| Example 1-1 | ETM 1 | 1.3754 | 7.56 | 183 | 9.39 |
| Example 1-2 | ETM 2 | 1.3887 | 7.70 | 167 | 9.37 |
| Example 1-3 | ETM 3 | 1.41 | 7.49 | 183 | 9.32 |
| Example 1-4 | ETM 4 | 1.421 | 7.42 | 167 | 9.09 |
| Example 1-5 | ETM 5 | 1.4275 | 7.42 | 169 | 9.11 |
| Example 1-6 | ETM 6 | 1.4386 | 7.29 | 208 | 9.37 |
| Example 1-7 | ETM 7 | 1.5587 | 7.36 | 175 | 9.11 |
| Example 1-8 | ETM 8 | 1.5844 | 7.70 | 153 | 9.23 |
| Example 1-9 | ETM 9 | 1.598 | 7.56 | 145 | 9.01 |
| Comparative | X2 | 1.6918 | 7.63 | 134 | 8.97 |
| Example 1-2 | | | | | |

Referring to Table 5, the organic light emitting devices according to Examples, each employing the electron transport material having a heterogeneous electron transfer rate constant (K) value within the range of 1.2 to 1.65, were found to have the evaluation criteria value of 9 or more, unlike the devices according to Comparative Examples, each employing the electron transport materials having a heterogeneous electron transfer rate constant (K) value out of the above range, indicating an excellent balance between current efficiency and lifetime.

### Comparative Example 2-1

As an anode, a substrate on which ITO was deposited at 30 Å was cut to a size of 50 mm×50 mm×0.5 mm, and immersed in distilled water in which a detergent was dissolved, and washed by ultrasonic waves. As a detergent, a product available from Fisher Co. was used. As the distilled water, distilled water filtered twice by using a filter available from Millipore Co. was used. The ITO was washed for 30 minutes, and ultrasonic washing was then repeated twice for 10 minutes by using distilled water. After the completion of washing with distilled water, ultrasonic washing was performed using isopropyl alcohol, acetone, and methanol solvent in this order, followed by drying.

A compound HTL1 and P1 were vacuum-deposited at a weight ratio of 97:3 on the prepared anode to form a hole injection layer in a thickness of 106 Å. Then, the compound HTL1 was vacuum-deposited on the hole injection layer in a thickness of 1000 Å to form a hole transport layer. Then, a compound HTL2 was vacuum-deposited on the hole transport layer in a thickness of 40 Å to form an electron blocking layer.

Next, a host BH and a dopant BD were vacuum-deposited at a weight ratio of 97:3 on the electron blocking layer to form a light emitting layer in a thickness of 190 Å.

Then, a hole blocking material Y1 was deposited on the light emitting layer in a thickness of 50 Å to form a hole blocking layer. Then, electron transport material ETM1 and LiQ were vacuum-deposited at a weight ratio of 50:50 to form an electron transport layer in a thickness of 250 Å. Subsequently, LiQ with a thickness of 7 Å was used to form an electron injection layer, and magnesium and silver (10:1) were used to form a cathode in a thickness of 100 Å. A capping layer (CPL) was deposited in a thickness of 800 Å to complete a device.

In the above process, a deposition rate of the organic material was maintained at 1 Å/sec. At this time, the vacuum deposition of each layer was performed using a cluster-type 1.0E-7 vacuum evaporator (manufactured by Selcos).

### Comparative Example 2-2 and Examples 2-1 to 2-8

Each organic light emitting device was manufactured in the same manner as in Comparative Example 2-1, except that each of the materials described in the following Table 6 was used as the hole blocking material.

The compounds used in Examples and Comparative Examples are the same as described above.

### Experimental Example 4: Characterization of Device According to k_{d} (Donating k) Value of Hole Blocking Material

Current efficiency and lifetime (T95) were measured using PR-655 IVL available from Photo Research, when current was applied to the organic light emitting devices manufactured in Examples and Comparative Examples, and the results are shown in the following Table 6. At this time, lifetime (T95) means the time to be taken until the luminance reaches 95% of the initial luminance.

Further, to evaluate a balance between current efficiency and lifetime of the organic light emitting devices according to k_{d} values of the hole blocking materials, evaluation criteria (α) was calculated by considering ranges of the efficiency and lifetime values as in the Equation 1, and the results are shown in the following Table 6.

TABLE 6

| | Kind | k_{d} value | Current | | Evalu- |
|---|---|---|---|---|---|
| | of hole | of hole | efficiency | T95 | ation |
| | blocking | blocking | (cd/A@10 | (hr@950 | criteria |
| | material | material | mA/cm²) | nit) | (α) |
| Comparative | Y1 | 1.2220 | 6.42 | 17 | 6.59 |
| Example 2-1 | | | | | |
| Example 2-1 | HBM 1 | 1.3685 | 6.28 | 320 | 9.48 |
| Example 2-2 | HBM 2 | 1.4178 | 6.15 | 337 | 9.52 |
| Example 2-3 | HBM 3 | 1.5993 | 5.88 | 357 | 9.45 |
| Example 2-4 | HBM 4 | 1.7641 | 6.89 | 229 | 9.18 |
| Example 2-5 | HBM 5 | 1.8571 | 6.82 | 220 | 9.02 |
| Example 2-6 | HBM 6 | 1.8453 | 6.42 | 263 | 9.05 |
| Example 2-7 | HBM 7 | 1.8464 | 6.35 | 289 | 9.24 |
| Example 2-8 | HBM 8 | 2.1158 | 6.89 | 232 | 9.21 |
| Comparative | Y2 | 2.7261 | 7.89 | 76 | 8.65 |
| Example 2-2 | | | | | |

Referring to Table 6, the organic light emitting devices according to Examples, each employing the hole blocking material having an electron donating rate constant k_{d} (donating k) within the range of 1.25 to 2.25, were found to have the evaluation criteria value of 9 or more, unlike the devices according to Comparative Examples, each employing the hole blocking material having an electron donating rate constant k_{d} (donating k) out of the above range, indicating an excellent balance between current efficiency and lifetime.

### REFERENCE NUMERALS

| | |
|---|---|
| 1: Substrate | 2: Anode |
| 3: Hole transport layer | 4: Light emitting layer |
| 5: Electron transport layer | 6: Cathode |
| 7: Hole injection layer | 8: Electron blocking layer |
| 9: Hole blocking layer | 10: Electron injection layer |

1. An organic light emitting device, comprising:
an anode;
a cathode positioned opposite to the anode;
a light emitting layer positioned between the anode and the cathode;
a hole transport layer positioned between the anode and the light emitting layer; and
an electron transport layer positioned between the light emitting layer and the cathode,
wherein the electron transport layer includes a metal complex compound and an electron transport material having a heterogeneous electron transfer rate constant (K) of 1.2 to 1.65, and
the heterogeneous electron transfer rate constant (K) is calculated by the following Mathematical Equation 1:
$$[ < Mathematical Equation 1 > \\ K = \frac{{k}_{d} + {k}_{a}}{2}$$
wherein in Mathematical Equation 1:
k_{d} (donating k) is an electron donating rate constant and kₐ (accepting k) is an electron accepting rate constant.

2. The organic light emitting device of claim 1, further comprising a hole blocking layer positioned between the light emitting layer and the electron transport layer,
wherein the hole blocking layer includes a hole blocking material having an electron donating rate constant k_{d} (donating k) of 1.25 to 2.25.

3. The organic light emitting device of claim 2, wherein the hole blocking layer is in contact with the light emitting layer.

4. The organic light emitting device of claim 1, wherein the electron transport material is a compound of Chemical Formula 1: 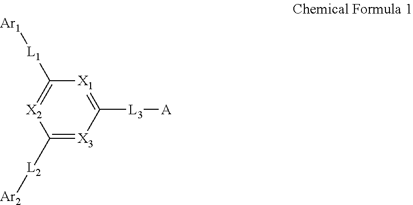
wherein in Chemical Formula 1:
X₁ to X₃ are each independently N or CH, and at least one of X₁ to X₃ is N;
L₁ to L₃ are each independently a single bond or a substituted or unsubstituted C₆₋₆₀ arylene;
Ar₁ and Ar₂ are each independently a substituted or unsubstituted C₆₋₆₀ aryl or a substituted or unsubstituted C₂₋₆₀ heteroaryl containing any one or more heteroatoms selected from the group consisting of N, O, and S;
A is a monovalent substituent of any one of the following Chemical Formulae 2-1 to 2-3; 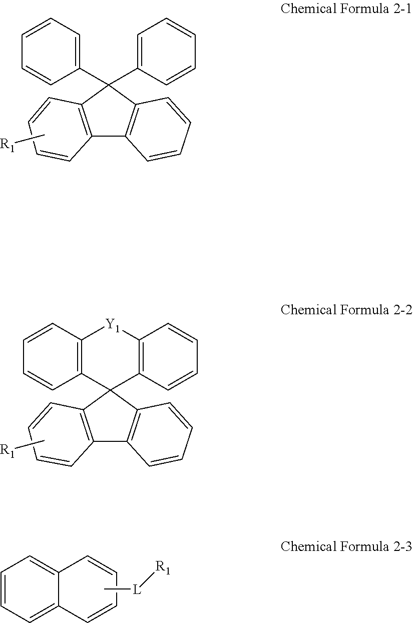
wherein in Chemical Formulae 2-1 to 2-3:
Y₁ is O or S;
L is a C₆₋₆₀ arylene; and
R₁ is hydrogen, deuterium, cyano, a C₆₋₆₀ aryl, or a C₆₋₆₀ aryl substituted with cyano.

5. The organic light emitting device of claim 4, wherein:
all of X₁ to X₃ are N; or
X₁ and X₂ are N, and X₃ is CH.

6. The organic light emitting device of claim 4, wherein:
L₁ to L₃ are each independently a single bond or phenylene; and
L is phenylene or naphthylene.

7. The organic light emitting device of claim 4, wherein Ar₁ and Ar₂ are each independently phenyl, biphenylyl, terphenylyl, naphthyl, or pyridinyl.

8. The organic light emitting device of claim 4, wherein A is any one substituent selected from the group consisting of the following substituents: 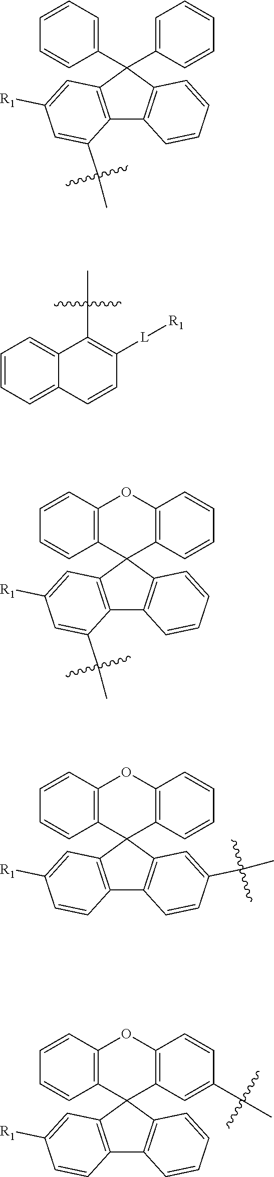
wherein L is naphthylene; and
R₁ is hydrogen, cyano, or cyanophenyl.

9. The organic light emitting device of claim 1, wherein the electron transport material is any one compound selected from the group consisting of the following compounds:

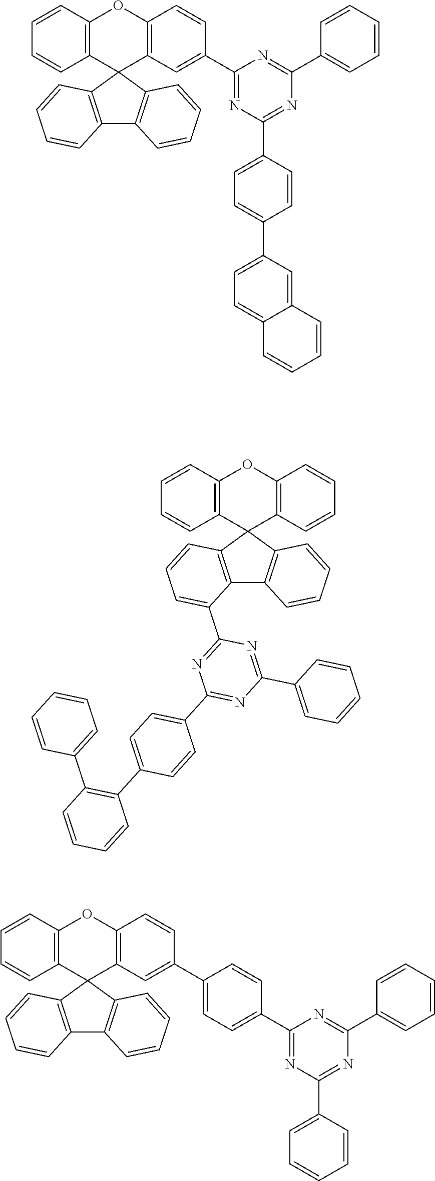 [SMILES: c1ccc(-c2nc(-c3ccc(-c4ccc5ccccc5c4)cc3)nc(-c3ccc4c(c3)C3(c5ccccc5O4)c4ccccc4-c4ccccc43)n2)cc1 ; c1ccc(-c2nc(-c3ccc(-c4ccccc4-c4ccccc4)cc3)nc(-c3cccc4c3-c3ccccc3C43c4ccccc4Oc4ccccc43)n2)cc1 ; c1ccc(-c2nc(-c3ccccc3)nc(-c3ccc(-c4ccc5c(c4)C4(c6ccccc6O5)c5ccccc5-c5ccccc54)cc3)n2)cc1]

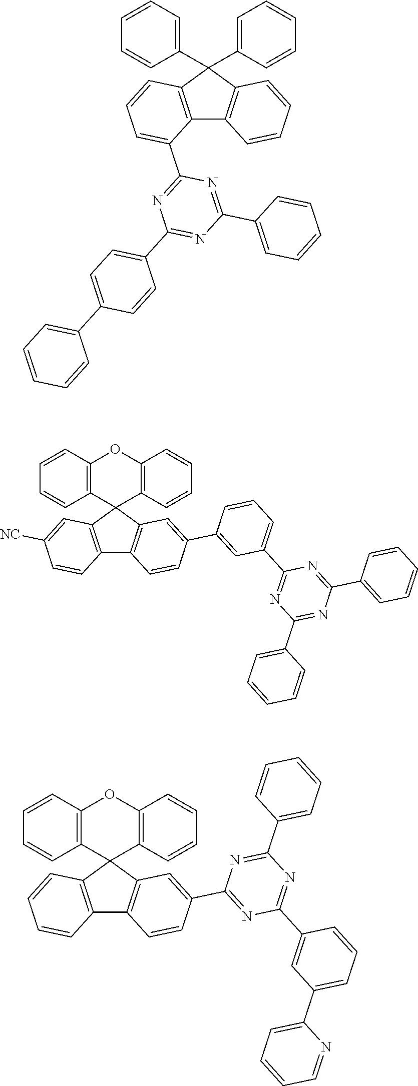 [SMILES: c1ccc(-c2ccc(-c3nc(-c4ccccc4)nc(-c4cccc5c4-c4ccccc4C5(c4ccccc4)c4ccccc4)n3)cc2)cc1 ; N#Cc1ccc2c(c1)C1(c3ccccc3Oc3ccccc31)c1cc(-c3cccc(-c4nc(-c5ccccc5)nc(-c5ccccc5)n4)c3)ccc1-2 ; c1ccc(-c2nc(-c3cccc(-c4ccccn4)c3)nc(-c3ccc4c(c3)C3(c5ccccc5Oc5ccccc53)c3ccccc3-4)n2)cc1]

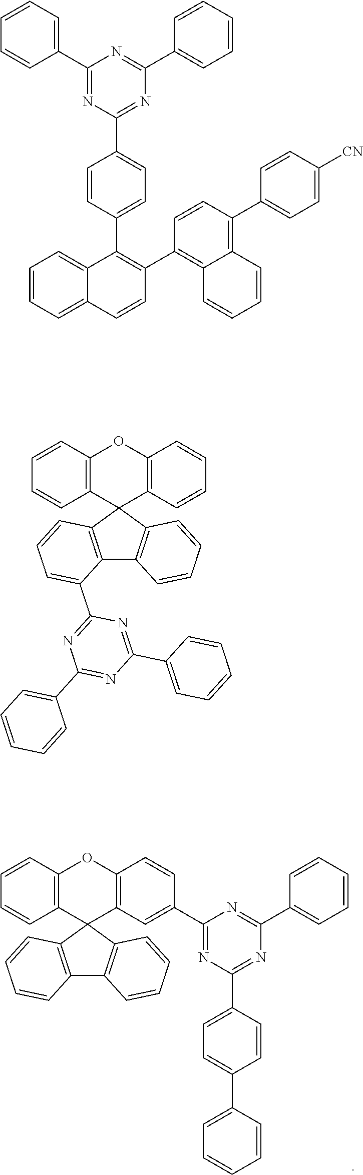 [SMILES: N#Cc1ccc(-c2ccc(-c3ccc4ccccc4c3-c3ccc(-c4nc(-c5ccccc5)nc(-c5ccccc5)n4)cc3)c3ccccc23)cc1 ; c1ccc(-c2nc(-c3ccccc3)nc(-c3cccc4c3-c3ccccc3C43c4ccccc4Oc4ccccc43)n2)cc1 ; c1ccc(-c2ccc(-c3nc(-c4ccccc4)nc(-c4ccc5c(c4)C4(c6ccccc6O5)c5ccccc5-c5ccccc54)n3)cc2)cc1]

10. The organic light emitting device of claim 1, wherein the metal complex compound is a compound of Chemical Formula 3:
M(L₁₁)ₙ₁(L₁₂)ₙ₂ Chemical Formula 3
wherein in Chemical Formula 3:
M is lithium, beryllium, manganese, copper, zinc, aluminum, or gallium;
L₁₁ is a substituted or unsubstituted 8-hydroxyquinolinato or a substituted or unsubstituted 10-hydroxybenzo[h]quinolinato;
L₁₂ is halogen, substituted or unsubstituted phenolato, or substituted or unsubstituted naphtholato;
n1 is 1, 2, or 3;
n2 is 0 or 1; and
n1+n2 is 1, 2, or 3.

11. The organic light emitting device of claim 10, wherein the metal complex compound is any one selected from the group consisting of 8-hydroxyquinolinato lithium, bis(8-hydroxyquinolinato)zinc, bis(8-hydroxyquinolinato)copper, bis(8-hydroxyquinolinato)manganese, tris(8-hydroxyquinolinato)aluminum, tris(2-methyl-8-hydroxyquinolinato)aluminum, tris(8-hydroxyquinolinato)gallium, bis(10-hydroxybenzo[h]quinolinato)beryllium, bis(10-hydroxybenzo[h]quinolinato)zinc, bis(2-methyl-8-hydroxyquinolinato)chlorogallium, bis(2-methyl-8-hydroxyquinolinato)(o-cresolato)gallium, bis(2-methyl-8-hydroxyquinolinato)(1-naphtholato)-, and bis(2-methyl-8-hydroxyquinolinato)(2-naphtholato)gallium.

12. The organic light emitting device of claim 1, wherein the electron transport layer includes the electron transport material and the metal complex compound at a weight ratio in a range of 70:30 to 30:70.

13. The organic light emitting device of claim 2, wherein the hole blocking material is a compound of Chemical Formula 4: 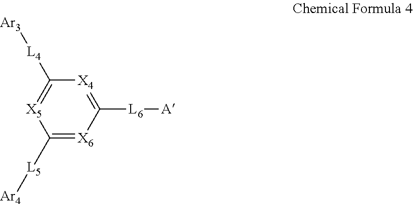
wherein in Chemical Formula 4:
X₄ to X₆ are each independently N or CH, wherein at least one of X₄ to X₆ is N;
L₄ to L₆ are each independently a single bond or a substituted or unsubstituted C₆₋₆₀ arylene;
Ar₃ and Ar₄ are each independently a substituted or unsubstituted C₆₋₆₀ aryl;
A′ is a monovalent substituent of a compound of Chemical Formula 5-1: 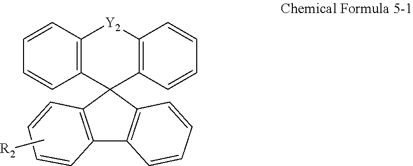
wherein in Chemical Formula 5-1:
Y₂ is O or S; and
R₂ is hydrogen, deuterium, cyano, a C₆₋₆₀ aryl, or a C₆₋₆₀ aryl substituted with cyano.

14. The organic light emitting device of claim 13, wherein all of X₄ to X₆ are N.

15. The organic light emitting device of claim 13, wherein L₄ to L₆ are each independently a single bond, phenylene, or biphenyldiyl.

16. The organic light emitting device of claim 13, wherein Ar₃ and Ar₄ are each independently phenyl, biphenylyl, terphenylyl, or naphthyl.

17. The organic light emitting device of claim 13, wherein A′ is any one substituent selected from the group consisting of the following substituents: 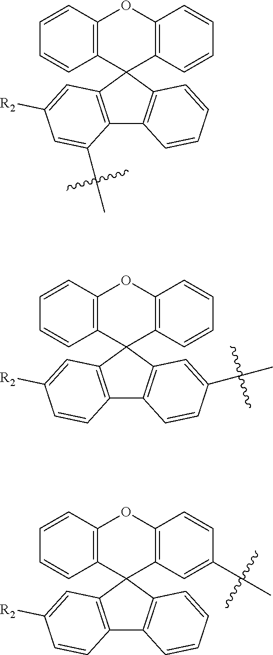
wherein R₂ is hydrogen or cyano.

18. The organic light emitting device of claim 2, wherein the hole blocking material is any one compound selected from the group consisting of the following compounds: 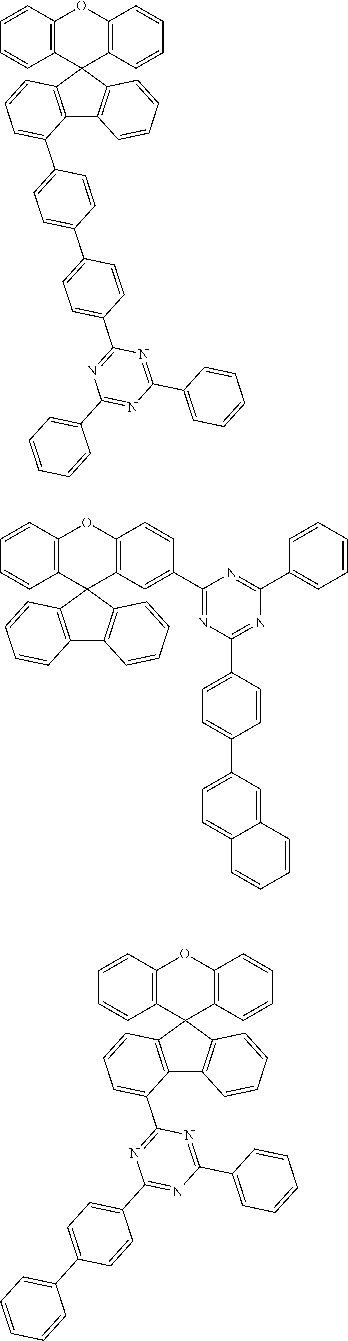 [SMILES: c1ccc(-c2nc(-c3ccccc3)nc(-c3ccc(-c4ccc(-c5cccc6c5-c5ccccc5C65c6ccccc6Oc6ccccc65)cc4)cc3)n2)cc1 ; c1ccc(-c2nc(-c3ccc(-c4ccc5ccccc5c4)cc3)nc(-c3ccc4c(c3)C3(c5ccccc5O4)c4ccccc4-c4ccccc43)n2)cc1 ; c1ccc(-c2ccc(-c3nc(-c4ccccc4)nc(-c4cccc5c4-c4ccccc4C54c5ccccc5Oc5ccccc54)n3)cc2)cc1] 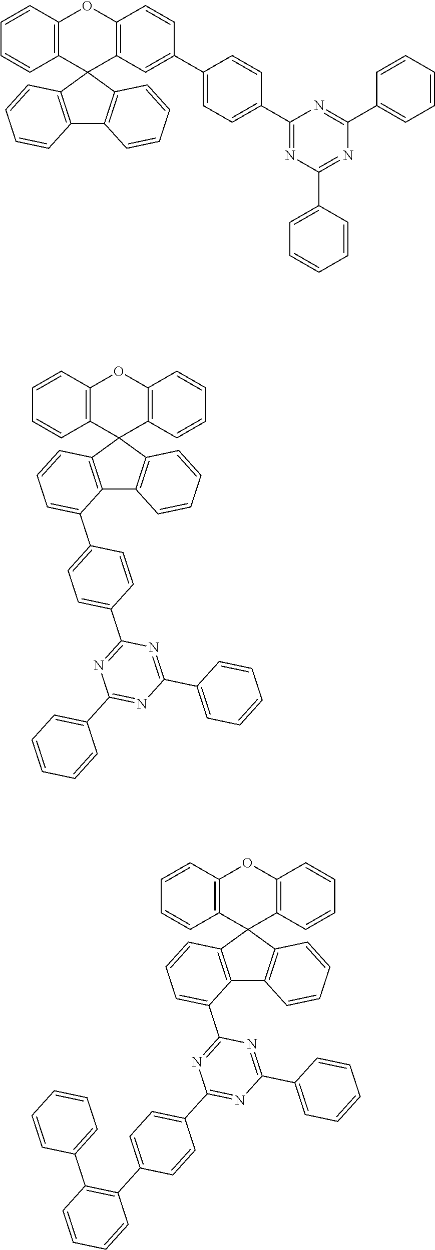 [SMILES: c1ccc(-c2nc(-c3ccccc3)nc(-c3ccc(-c4ccc5c(c4)C4(c6ccccc6O5)c5ccccc5-c5ccccc54)cc3)n2)cc1 ; c1ccc(-c2nc(-c3ccccc3)nc(-c3ccc(-c4cccc5c4-c4ccccc4C54c5ccccc5Oc5ccccc54)cc3)n2)cc1 ; c1ccc(-c2nc(-c3ccc(-c4ccccc4-c4ccccc4)cc3)nc(-c3cccc4c3-c3ccccc3C43c4ccccc4Oc4ccccc43)n2)cc1] 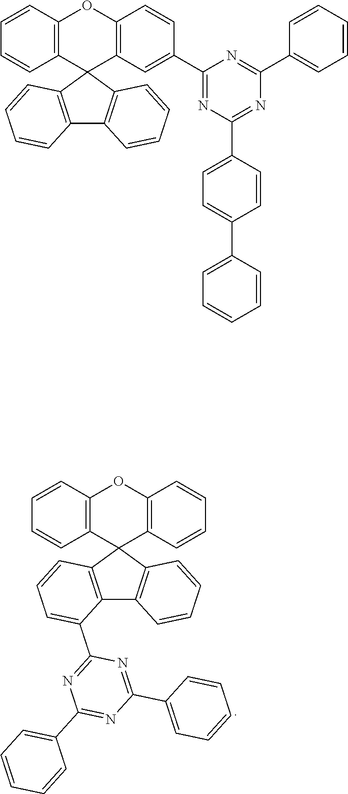 [SMILES: c1ccc(-c2ccc(-c3nc(-c4ccccc4)nc(-c4ccc5c(c4)C4(c6ccccc6O5)c5ccccc5-c5ccccc54)n3)cc2)cc1 ; c1ccc(-c2nc(-c3ccccc3)nc(-c3cccc4c3-c3ccccc3C43c4ccccc4Oc4ccccc43)n2)cc1]
